(19) United States
(12) Patent Application Publication
(10) Pub. No.: US 2026/0041501 A1
(43) Pub. Date: Feb. 12, 2026

(54) DRIVE ASSEMBLY FOR ROBOTIC SURGICAL INSTRUMENT

(71) Applicant: Auris Health, Inc., Redwood City, CA (US)

(72) Inventors: Thomas R. Jenkins, Alameda, CA (US); Luis Andrade Baez, Mountain View, CA (US); James Hayes, La Honda, CA (US)

(21) Appl. No.: 18/799,516

(22) Filed: Aug. 9, 2024

(51) Int. Cl. A61B 34/30; A61B 34/00; A61M 25/01

(52) U.S. Cl. CPC A61B 34/30; A61B 2034/303; A61B 2034/715; A61M 25/0113

(57) ABSTRACT

An apparatus includes a housing and a first elongate member extending distally from the housing to a first distal end. A proximal portion of the first elongate member defines a longitudinal axis. The apparatus also includes a first drive input rotatably retained by the housing. The first drive input is configured to deflect the first distal end relative to the longitudinal axis. The apparatus further includes a second elongate member. The apparatus also includes a second drive input rotatably retained by the housing. The apparatus further includes a feed assembly configured to translate the second elongate member along the longitudinal axis in response to rotation of the second drive input.

### BACKGROUND

A variety of surgical instruments include an end effector for use in medical treatments and procedures conducted by a medical professional operator, including applications in robotically assisted surgeries. In the case of robotically assisted surgery, the surgeon may operate a primary controller to remotely control the motion of such surgical instruments at a surgical site. The controller may be separated from the patient by a significant distance (e.g., across the operating room, in a different room, or in a completely different building than the patient); or quite near the patient in the operating room. The controller may include one or more hand input devices (e.g., joysticks, exoskeletal gloves, primary manipulators, etc.), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including a biopsy sample acquisition instrument, a kidney stone removal instrument, a viewing scope, an ultrasonic blade, a surgical stapler, a tissue grasper, a needle driver, an electrosurgical cautery probe, etc. Each of these structures performs functions for the surgeon, for example, obtaining a biopsy sample, removing a kidney stone or other structure, observing structures and other features within a patient, cutting tissue, coagulating tissue, manipulating a needle, grasping a blood vessel, dissecting tissue, or cauterizing tissue. A robotically controlled instrument may be introduced into the patient via an incision, via a naturally occurring orifice, or otherwise.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a top plan view of an example of a robotic surgical system being used in a urological procedure;

FIG. 2 depicts a schematic view of different components of the robotic surgical system of FIG. 1;

FIG. 3 depicts enlarged views of other components of the robotic surgical system of FIG. 1, including a distal portion of a ureteroscope;

FIG. 4 depicts a perspective view of an example of an instrument drive mechanism that may be used with the robotic surgical system of FIG. 1;

FIG. 5 depicts a top plan view of the instrument drive mechanism of FIG. 4;

FIG. 6 depicts a perspective view of an example of an instrument handle that may be used with the robotic surgical system of FIG. 1;

FIG. 7 depicts a bottom plan view of the instrument handle of FIG. 6;

FIG. 8A depicts a top perspective view of another example of an instrument handle that may be used with the robotic surgical system of FIG. 1 to steer a first elongate member, and having a proximal feed assembly that is configured to longitudinally translate a second elongate member, showing the second elongate member in a proximally retracted position;

FIG. 8B depicts a top perspective view of the instrument handle of FIG. 8A, showing the second elongate member in a distally extended position;

FIG. 9 depicts a rear perspective view of the instrument handle of FIG. 8A;

FIG. 10A depicts a top perspective view of another example of an instrument handle that may be used with the robotic surgical system of FIG. 1 to steer a first elongate member and/or a second elongate member, and having an internal feed assembly that is configured to longitudinally translate the second elongate member, showing the first elongate member in a straight configuration, and further showing the second elongate member in a proximally retracted position and in a straight configuration;

FIG. 10B depicts a top perspective view of the instrument handle of FIG. 10A, showing the first elongate member in a vertically deflected configuration, and further showing the second elongate member in a proximally retracted position and in a straight configuration;

FIG. 10C depicts a top perspective view of the instrument handle of FIG. 10A, showing the first elongate member in a laterally deflected configuration, and further showing the second elongate member in a proximally retracted position and in a straight configuration;

FIG. 10D depicts a top perspective view of the instrument handle of FIG. 10A, showing the first elongate member in the straight configuration, and further showing the second elongate member in a distally extended position and in the straight configuration;

FIG. 10E depicts a top perspective view of the instrument handle of FIG. 10A, showing the second elongate member in the distally extended position and in a vertically deflected configuration; and

FIG. 10F depicts a top perspective view of the instrument handle of FIG. 10A, showing the second elongate member in the distally extended position and in a laterally deflected configuration.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

The term “substantially” is also utilized herein to represent the degree by which a quantitative representation may vary from a stated reference without resulting in a change in the basic function of the subject matter at issue. The term “substantially” shall therefore be understood to include a range of conditions or results that provide a functional equivalent to an explicitly stated condition or result. As a non-limiting example, an apparatus including a component that is “substantially straight” or “substantially flat” may provide a result or effect that is functionally equivalent to a result or effect that would be achieved by the same apparatus including the same component in a perfectly straight or perfectly flat configuration. The range implied by the term “substantially” should also be read to include the perfect result that is within that range. Thus, terms such as “substantially straight” and “substantially flat” shall be read as including “perfectly straight” and “perfectly flat,” respectively; while also including a range of straightness or flatness that is functionally equivalent to perfectly straight or flat, respectively. As with the terms “approximately” and “about,” the term “substantially” may indicate a suitable dimensional tolerance, or other form of reasonable expected range, that allows a part or collection of components to function for its intended purpose as described herein.

Aspects of the present examples described herein may be integrated into a robotically enabled medical system, including as a robotic surgical system, capable of performing a variety of medical procedures, including both minimally invasive, such as laparoscopy, and non-invasive, such as endoscopy, procedures. Among endoscopy procedures, the robotically enabled medical system may be capable of performing bronchoscopy, ureteroscopy, gastroscopy, etc.

In addition to performing the breadth of procedures, the robotically enabled medical system may provide additional functionalities, such as enhanced imaging and guidance to assist the medical professional. Additionally, the robotically enabled medical system may provide the medical professional with the ability to perform the procedure from an ergonomic position without the need for awkward arm motions and positions. Still further, the robotically enabled medical system may provide the medical professional with the ability to perform the procedure with improved ease of use such that one or more of the instruments of the robotically enabled medical system may be controlled by a single operator.

### I. Example of Robotically Enabled Medical System

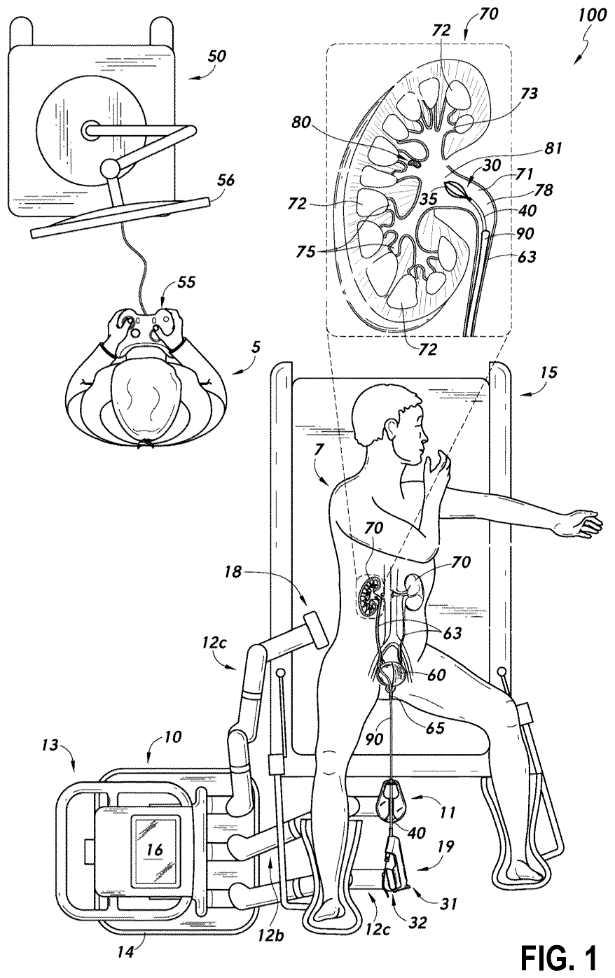

FIG. 1 shows an example medical system (100) for performing various medical procedures in accordance with aspects of the present disclosure. The medical system (100) may be used in, for example, endoscopic (e.g., ureteroscopic) procedures. Certain ureteroscopic procedures involve the treatment/removal of kidney stones. Although the system (100) of FIG. 1 is presented in the context of a ureteroscopic procedure, the principles disclosed herein may be implemented in any type of endoscopic (e.g., bronchial, gastrointestinal, etc.) and/or percutaneous procedure.

The medical system (100) of the present includes a robotic system (10) (e.g., mobile robotic cart) that is configured to engage with and/or control one or more medical instruments (e.g., ureteroscope (40), basketing system (30), etc.) via one or more robotic arms (12) to perform a direct-entry procedure on a patient (7). In some versions, the robotic system (10) and/or control system (50) is/are configured to receive images and/or image data from the scope (40) representing internal anatomy of the patient (7), namely the urinary system with respect to the depiction of FIG. 1, and/or display images based thereon.

It should be understood that the direct-entry instrument(s) operated through systems (10, 50) may include any type of medical instrument or combination of instruments, including an endoscope (such as a ureteroscope (40)), catheter (such as a steerable or non-steerable catheter), nephroscopes, laparoscope, basketing systems (30), and/or other type of medical instrument(s). The various scope-type instruments disclosed herein, such as the scope (40) of the system (100), may be configured to navigate within the human anatomy, such as within a natural orifice or lumen of the human anatomy; or via a formed opening or other portal in the human body. The terms “scope” and “endoscope” are used herein according to their broad and ordinary meanings; and may refer to any type of elongate medical instrument having image generating, viewing, and/or capturing functionality and configured to be introduced into any type of organ, cavity, lumen, chamber, or space of a body. A scope may include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise a rigid or flexible tube, and may be dimensioned to be passed within an outer sheath, catheter, introducer, or other lumen-type device, or may be used without such devices.

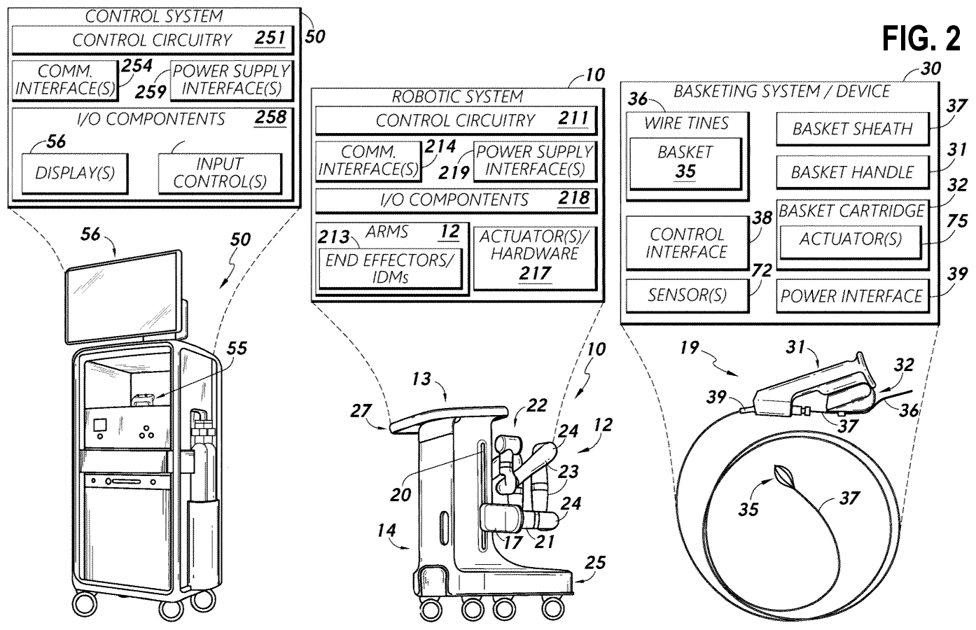

The medical system (100) of the present example further includes a control system (50), a table (15), and an electromagnetic (EM) field generator (18). Table (15) is configured to hold the patient (7). EM field generator (18) may be held by one or more of the robotic arms (12) of the robotic system (10) or may be a stand-alone device. As shown in FIGS. 1-2, control system (50) of the present example includes various input/output (I/O) components (258) configured to assist the physician (5) or others in performing a medical procedure. For example, the I/O components (258) may be configured to allow for user input to control/navigate the scope (40) and/or basketing system (30) within the patient (7). I/O components (258) of the present example include a controller (55) that is configured to receive user input from the operator; and a display (56) that is configured to present certain information to assist the operator. Controller (55) may take any suitable form, including but not limited to one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, etc.

As also shown in FIG. 2, control system (50) of the present example includes a communication interface (254) that is operable to provide a communicative interface between control system (50) and robotic system (10), basketing system (30), scope (40), and/or other components. Communications via communication interface (254) may include data, commands, electrical power, and/or other forms of communication. Communication interface (254) may also be configured to provide communication via wire, wirelessly, and/or other modalities. Control system (50) also includes a power supply interface (259), which may receive power to drive control system (50) via wire, battery, and/or any other suitable kind of power source. Control circuitry (251) of control system (50) may provide signal processing and execute control algorithms to achieve the functionality of medical system (100) as described herein.

The control system (50) may also communicate with the robotic system (10) to receive position data therefrom relating to the position of the distal end of the scope (40), access sheath (90), or basketing device (30). Such positional data relating to the position of the scope (40), access sheath (90), or basketing device (30) may be derived using one or more electromagnetic sensors associated with the respective components. Moreover, in some versions, the control system (50) may communicate with the table (15) to position the table (15) in a particular orientation or otherwise control the table (15). The control system (50) may also communicate with the EM field generator (18) to control generation of an EM field in an area around the patient (7).

As noted above and as shown in FIGS. 1-2, robotic system (10) includes robotic arms (12) that are configured to engage with and/or control scope (40) and/or the basketing system (30) to perform one or more aspects of a procedure. Robotic arms (12) may alternatively be coupled to different instruments than what is shown in FIG. 1; and in some scenarios, one or more of the robotic arms (12) may not be utilized or coupled to a medical instrument. Each robotic arm (12) includes multiple arm segments (23) coupled to joints (24), which may provide multiple degrees of movement/freedom. In the example of FIG. 1, the robotic system (10) is positioned proximate to the patient's legs and the robotic arms (12) are actuated to engage with and position the scope (40) for access into an access opening, such as the urethra (65) of the patient (7). When the robotic system (10) is properly positioned, the scope (40) may be inserted into the patient (7) robotically using the robotic arms (12), manually by the physician (5), or a combination thereof. A scope-driver instrument coupling (11) (e.g., instrument device manipulator (IDM)) may be attached to the distal portion of one of the arms (12b) to facilitate robotic control/advancement of the scope (40). Another (12c) of the arms may include an instrument coupling/manipulator (19) that is configured to facilitate advancement and operation of the basketing device (30). The scope (40) may include one or more working channels through which additional tools, such as lithotripters, basketing devices, forceps, etc., may be introduced into the treatment site.

The robotic system (10) may be coupled to any component of the medical system (100), such as the control system (50), the table (15), the EM field generator (18), the scope (40), the basketing system (30), and/or any type of percutaneous-access instrument (e.g., needle, catheter, nephroscope, etc.). As noted above, robotic system (10) may be communicatively coupled with control system (50) via communication interfaces (214, 254). Robotic system (10) also includes a power supply interface (219), which may receive power to drive robotic system (10) via wire, battery, and/or any other suitable kind of power source. In addition, robotic system (10) of the present example includes various input/output (I/O) components (218) configured to assist the physician (5) or others in performing a medical procedure. Such I/O components (218) may include any of the various kinds of I/O components (258) described herein in the context of control system (50). In addition, or in the alternative, I/O components (218) of robotic system (10) may take any suitable form (or may be omitted altogether).

Robotic system (10) of the present example generally includes a column (14), a base (25), and a console (13) at the top of the column (14). The column (14) may include one or more arm supports (17) (also referred to as a “carriage”) for supporting the deployment of the one or more robotic arms (12) (three shown in FIG. 2). The arm support (17) may include individually configurable arm mounts that rotate along a perpendicular axis to adjust the base of the robotic arms (12) for desired positioning relative to the patient. In some versions, the arm support (17) may be connected to the column (14) through slots (20) that are positioned on opposite sides of the column (14) to guide vertical translation of the arm support (17) along column (14). The robotic arms (12) of the present example generally comprise robotic arm bases (21) and end effectors (22), separated by a series of linking arm segments (23) that are connected by a series of joints (24), each joint comprising one or more independent actuators (217). Each actuator (217) may comprise an independently controllable motor. I/O components (218) may be positioned at the upper end of column (14). Console (13) also includes a handle (27) to assist with maneuvering and stabilizing robotic system (10).

The end effector (213) of each of the robotic arms (12) may include an instrument device manipulator (IDM), which may be attached using a mechanism changer interface (MCI). In some versions, the IDM (213) may be removed and replaced with a different type of IDM (213), for example, a first type (11) of IDM (213) may manipulate a scope (40), while a second type (19) of IDM (213) may manipulate a basketing system (30). Another type of IDM (213) may be configured to hold an electromagnetic field generator (18). An MCI may provide power and control interfaces (e.g., connectors to transfer pneumatic pressure, electrical power, electrical signals, and/or optical signals from the robotic arm (12) to the IDM (213). The IDMs (213) may be configured to manipulate medical instruments (e.g., surgical tools/instruments), such as the scope (40), using techniques including, for example, direct drives, harmonic drives, geared drives, belts and pulleys, magnetic drives, and the like.

The system (100) may include certain control circuitry configured to perform certain of the functionality described herein, including the control circuitry (211) of the robotic system (10) and the control circuitry (251) of the control system (50). That is, the control circuitry of the system (100) may be part of the robotic system (10), the control system (50), or some combination thereof. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in versions in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry (211, 251) may comprise computer-readable media storing, and/or configured to store, hard-coded and/or operational instructions corresponding to at least some of the steps and/or functions illustrated in one or more of the present figures and/or described herein. Such computer-readable media may be included in an article of manufacture in some instances. The control circuitry (211, 251) may be entirely locally maintained/disposed or may be remotely located at least in part (e.g., communicatively coupled indirectly via a local area network and/or a wide area network).

In some versions, for example, the physician (5) may provide input to the control system (50) and/or robotic system (10); and in response to such input, control signals may be sent to the robotic system (10) to manipulate the scope (40) and/or catheter basketing system (30). The control system (50) may include one or more display devices (56) to provide various information regarding a procedure. For example, the display(s) (56) may provide information regarding the scope (40) and/or basketing system (30). The control system (50) may receive real-time images that are captured by the scope (40) and display the real-time images via the display(s) (56).

As shown in FIG. 2, the basketing device (30) of the present example includes a basket (35) formed of one or more wire tines (36) disposed within a basketing sheath (37) over a length thereof, where the tines project from a distal end of the sheath (37) to form the basket (35). The tines (36) further extend from the proximal end of the sheath (37) and are slidable within the basketing sheath (37). The tines (36) and the sheath (37) may be coupled to respective actuators (75) of a basket cartridge component (32). The basket cartridge (32) may be physically and/or communicatively coupled to a handle portion/component (31) of the basketing system (30). The handle component (31) may be configured to be used to assist in basketing control either manually or through robotic control. The basketing system (30) may be powered through a power interface (39) and/or controlled through a control interface (38), each or both of which may interface with a robotic arm/component of the robotic system (10). The basketing system (30) may further comprise one or more sensors (72), such as pressure and/or other force-reading sensors, which may be configured to generate signals indicating forces experienced at/by one or more of the actuators (75) and/or other couplings of the basketing system (30).

In an example of a use case, if the patient (7) has a kidney stone (80) located in the kidney (70), the physician may execute a procedure to remove the stone (80) through the urinary tract (65, 60, 63). In particular, and as shown in FIG. 1, the physician may operate the medical system (100) to achieve direct entry of the scope (40) into the urinary tract (65, 60, 63) of the patient (7) via the urethra (65). The physician (5) may interact with the control system (50) and/or the robotic system (10) to cause/control the robotic system (10) to advance and navigate the scope (40) from the urethra (65), through the bladder (60), up the ureter (63), and into the renal pelvis (71) and/or calyx network of the kidney (70) where the stone (80) is located. The physician (5) may further interact with the control system (50) and/or the robotic system (10) to cause/control the advancement of a basketing device (30) through a working channel of the scope (40), where the basketing device (30) is configured to facilitate capture and removal of a kidney stone. The control system (50) may provide information via the display(s) (56) that is associated with the medical instrument (40), such as real-time endoscopic images captured therewith, and/or other instruments of the system (100), to assist the physician (5) in navigating/controlling such instrumentation.

In the present example, a ureteral access sheath (90) is disposed within the urinary tract (65, 60, 63) to an area near the kidney (70). The scope (40) may be passed through the ureteral access sheath (90) to gain access to the internal anatomy of the kidney (70), as shown. Once at the site of the kidney stone (80) (e.g., within a target calyx (73) of the kidney (70) through which the stone (80) is accessible), the scope (40) may be used to channel/direct the basketing device (30) to the target location. Once the stone (80) has been captured in the distal basket portion (35) of the basketing device (30), the utilized ureteral access path may be used to extract the kidney stone (80) from the patient (7).

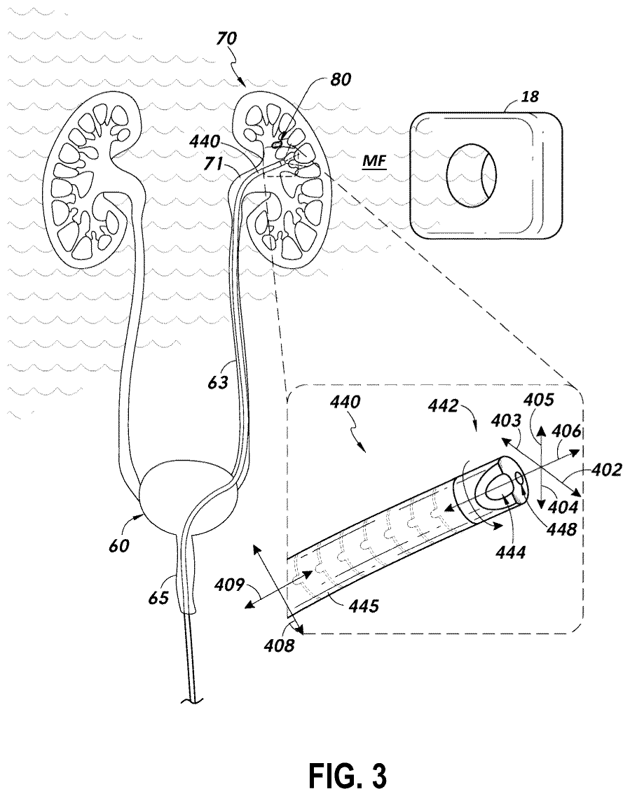

FIG. 3 shows an example of a scope (440) that may be used as scope (40) described above. Scope (440) of this example includes a working channel (444) for deploying medical instruments (e.g., lithotripters, basketing system (30), forceps, etc.), irrigation, and/or aspiration to an operative region at a distal end of the scope (440). The scope (440) may be articulated, such as with respect to at least a distal portion of the scope (440), so that the scope (440) can be steered within the human anatomy. In some versions, the scope (440) is configured to be articulated with, for example, five degrees of freedom, including XYZ coordinate movement, as well as pitch and yaw. In some versions, the scope (440) provides six degrees of freedom, including X, Y, and Z ordinate positions, as well as pitch, roll, and yaw. Position sensor(s) of the scope (440) may likewise have similar degrees of freedom with respect to the position information they produce/provide. As shown in FIG. 3, the tip (442) of the scope (440) may be oriented with zero deflection relative to a longitudinal axis (406) thereof (also referred to as a “roll axis”).

In the present example, the scope (440) can accommodate wires and/or optical fibers to transfer signals to/from an optical assembly and a distal end (442) of the scope (440), which can include an imaging device (448), such as an optical camera. The imaging device (448) may be used to capture images of an internal anatomical space, such as a target calyx/papilla of the kidney (70). The scope (440) may further be configured to accommodate optical fibers to carry light from proximately located light sources, such as light-emitting diodes, to the distal end (442) of the scope (440). The distal end (442) of the scope (440) may include ports for light sources to illuminate an anatomical space when using the imaging device (448). The imaging device (448) may comprise an optical fiber, fiber array, and/or lens; or a light-emitting diode at distal end (442). The optical components of imaging device (448) move along with the distal end (442) of the scope (440), such that movement of the distal end (442) of the scope (440) results in changes to the images captured by the imaging device(s) (448).

To capture images at different orientations of the tip (442), robotic system (10) may be configured to deflect the tip (442) on a positive yaw axis (402), negative yaw axis (403), positive pitch axis (404), negative pitch axis (405), or roll axis (406). The tip (442) or body (445) of the scope (440) may be elongated or translated in the longitudinal axis (406), x-axis (408), or y-axis (409). The scope (440) may include a reference structure (not shown) to calibrate the position of the scope (440). For example, robotic system (10) and/or control system (50) may measure deflection of the scope (440) relative to the reference structure. The reference structure may be located, for example, on a proximal end of the endoscope (440) and may include a key, slot, or flange.

A robotic arm (12) of robotic system (10) may be configured/configurable to manipulate the scope (440) as described above. Such manipulation may be performed by actuating one or more elongate members such as one or more pull wires (e.g., pull or push wires), cables, fibers, and/or flexible shafts. For example, robotic arms (12) may be configured to actuate multiple pull wires (not shown) coupled to the scope (440) to deflect the tip (442) of the scope (440). Pull wires may include any suitable or desirable materials, such as metallic and non-metallic materials such as stainless steel, aramid fiber, tungsten, carbon fiber, and the like. In some versions, the scope (440) is configured to exhibit nonlinear behavior in response to forces applied by the elongate movement members. The nonlinear behavior may be based on stiffness and compressibility of the scope (440), as well as variability in slack or stiffness between different elongate movement members.

In some versions, the scope (440) includes at least one sensor that is configured to generate and/or send sensor position data to another device. The sensor position data can indicate a position and/or orientation of the scope (440) (e.g., the distal end (442) thereof) and/or may be used to determine/infer a position/orientation of the scope (440). For example, a sensor (sometimes referred to as a “position sensor”) may include an electromagnetic (EM) sensor with a coil of conductive material or other form of an antenna. In some versions, the position sensor is positioned on the distal end (442) of scope (440), while in other examples the sensor is positioned at another location on scope (440).

As shown in FIG. 3, EM field generator (18) is configured to broadcast an alternating EM field 90 that is detected by the EM position sensor of scope (440). The alternating magnetic field (MF) induces small currents in coils of the EM position sensor, which may be analyzed to determine a distance and/or angle/orientation between the EM position sensor and the EM field generator (18). It should be understood that scope (440) may include other types of sensors, such as a shape sensing fiber, accelerometer(s), gyroscope(s), satellite-based positioning sensor(s) (e.g., global positioning system (GPS) sensors), radio-frequency transceiver(s), and so on. In the present example, the EM position sensor of scope (440) provides sensor data to control system (50), which is then used to determine a position and/or an orientation of scope (440).

### II. Examples of Instrument Device Manipulator and Instrument Handle

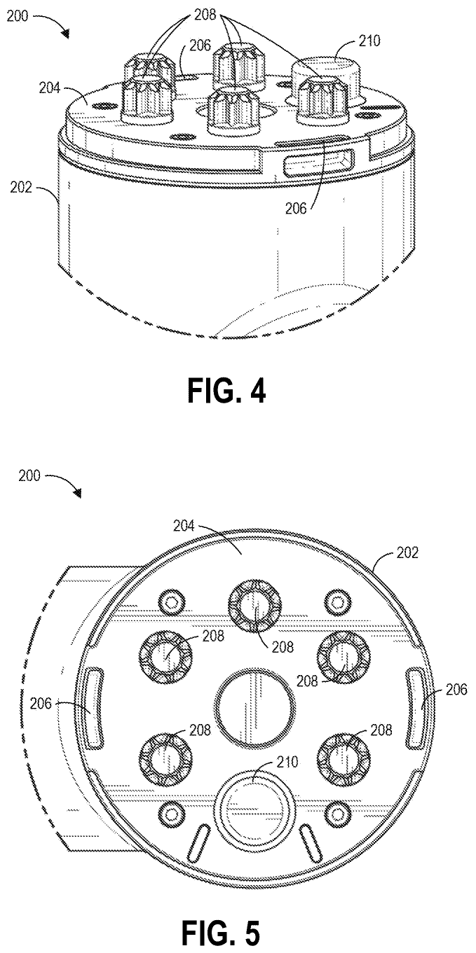

FIGS. 4-7 illustrate examples of an instrument device manipulator (also referred to as an instrument drive mechanism or IDM) (200) and an instrument handle (also referred to as an instrument base) (220) that may be used with robotic surgical system (10) to manipulate an elongate member, such as scope (40), access sheath (90), scope (440), a catheter (not shown), and/or any other suitable kind of elongate instrument. In other words, IDM (200) described below represents an example of a form that may be taken by IDM (213) described above. Instrument handle (220) described below represents an example of a form that may be taken by handle component (31) described above and/or by a handle of any of scope (40), access sheath (90), or scope (440) described above. While referred to as a “handle,” instrument handle (220) may not necessarily be grasped by a human hand at any point during operation of robotic surgical system (10).

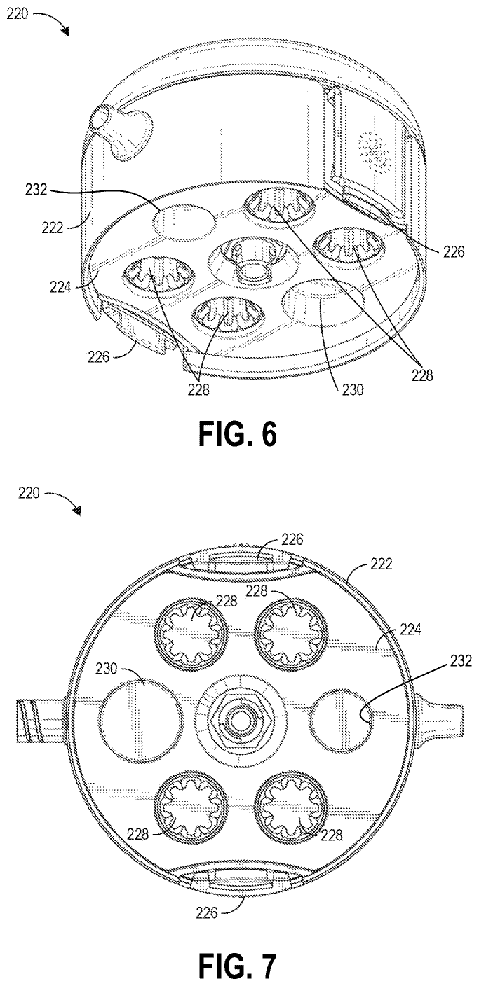

FIGS. 4 and 5 illustrate isometric and end views, respectively, of an example of an IDM (200) including a plurality of drive outputs (208) configured to engage a corresponding plurality of drive inputs (228) (FIGS. 6 and 7) of a medical instrument, such as a scope similar to the scopes (40, 440) described above. As illustrated, the IDM (200) includes a housing (202). The housing (202) includes an attachment face (204) that is configured to dock to the medical instrument. The IDM (200) includes latching mechanisms (206) that secure the medical instrument to the IDM (200) when docked.

As illustrated in FIGS. 4-5, the IDM (200) includes drive outputs (208). The drive outputs (208) can be, for example, rotatable elements configured to be driven by motors that are positioned, for example, within the housing (202). In the illustrated example, the drive outputs (208) are configured as splines or gears that protrude relative to the attachment face (204), although other configurations for the drive outputs (208) are possible. For example, the drive outputs (208) can comprise receptacles or sockets that are recessed relative to the attachment face (204). The drive outputs (208) are configured to couple with and engage corresponding drive inputs (228) (FIGS. 6-7) on the medical instrument. The drive outputs (208) transfer rotational motion (or other motion depending on the version) from the motors in the IDM (200) to the drive inputs (228) of the medical instrument such that the motors can be used to control the medical instrument during a procedure.

In some versions, the IDM (200) can include additional features, such as a communication module (210). The communication module (210) can be configured to read information from a medical instrument that is docked to the IDM (200). In some versions, the communication module (210) (or some other portion of the IDM (200)) comprises one or more proximity sensors. The proximity sensors may be configured to determine when a medical instrument is in the process of being docked to the IDM (200) or has been docked to the IDM (200). The proximity sensors may comprise one or more magnetic proximity sensors, for example.

FIGS. 6 and 7 illustrate isometric and end views, respectively, of an example of an instrument handle (220) of a medical instrument including a plurality of drive inputs (228) configured to engage the corresponding plurality of drive outputs (208) of the IDM (200). As illustrated, the instrument handle (220) includes a housing (222). The housing (222) includes an attachment face (224) that is configured to dock to the attachment face (204) of the IDM (200). The instrument handle (220) includes latching mechanisms (226) that secure the instrument handle (220) to the IDM (200) when docked.

As illustrated in FIGS. 6-7, the instrument handle (220) includes drive inputs (228). The drive inputs (228) can be rotatable elements configured to be driven by the drive outputs (208) of the IDM (200). In the illustrated example, the drive inputs (228) are configured as receptacles or sockets that are recessed relative to the attachment face (224), although other configurations for the drive inputs (228) are possible. For example, the drive inputs (228) can comprise protruding splines or gears that extend relative to the attachment face (224).

In some versions, the instrument handle (220) can include additional features, such as communication module (230), which can be configured to transmit information to communication module (210) of the IDM (200). In some versions, the communication module (230) (or some other portion of the instrument handle (220)) comprises one or more proximity sensors. The proximity sensors may be configured to determine when the medical instrument is in the process of being or has been docked to the IDM (200). The proximity sensors may comprise one or more magnetic proximity sensors.

### III. Examples of Instrument Handles With Proximal Drive Inputs

As noted above, IDM (200) may include a plurality of drive outputs (208) and instrument handle (220) may include a plurality of drive inputs (228), with each drive input (228) of instrument handle (220) being configured to be driven by a corresponding drive output (208) of IDM (200). In the examples shown in FIGS. 4-7, IDM (200) includes five drive outputs (208): a pair of distal drive outputs (208), a pair of intermediate drive outputs (208), and a single proximal drive output (208)); while instrument handle (220) includes four drive inputs (228): a pair of distal drive inputs (228) configured to be driven by the pair of distal drive outputs (208), and a pair of intermediate drive inputs (228) configured to be driven by the pair of intermediate drive outputs (208). Instrument handle (220) of the example shown also includes a proximal, substantially cylindrical bore (232) that lacks a proximal drive input (228), such that proximal bore (232) may be configured to accommodate the proximal drive output (208), but the proximal drive output (208) may not be configured to transfer rotational motion to proximal bore (232).

While the four drive inputs (228) of instrument handle (220) may be sufficient to perform one or more operations, such as steering the medical instrument (e.g., via corresponding pull wires), it may be desirable to configure instrument handle (220) to perform one or more additional operations that might otherwise need to be performed either manually or by a second instrument handle (220) (and corresponding second IDM (200)). For example, in instances where the medical device includes a first elongate member (e.g., a scope similar to scopes (40, 440)) that is steerable by the four drive inputs (228) of instrument handle (220), it may be desirable to configure instrument handle (220) to longitudinally translate a second elongate member (e.g., a sheath similar to access sheath (90), a biopsy tool shaft, etc.) over or through the first elongate member, without interfering with the ability of instrument handle (220) to steer the first elongate member. The following describes several examples of instrument handles with proximal drive inputs that are configured to utilize the proximal drive output (208) of IDM (200) to perform such additional operations.

### A. Example of Instrument Handle with Proximal Elongate Member Feed Assembly

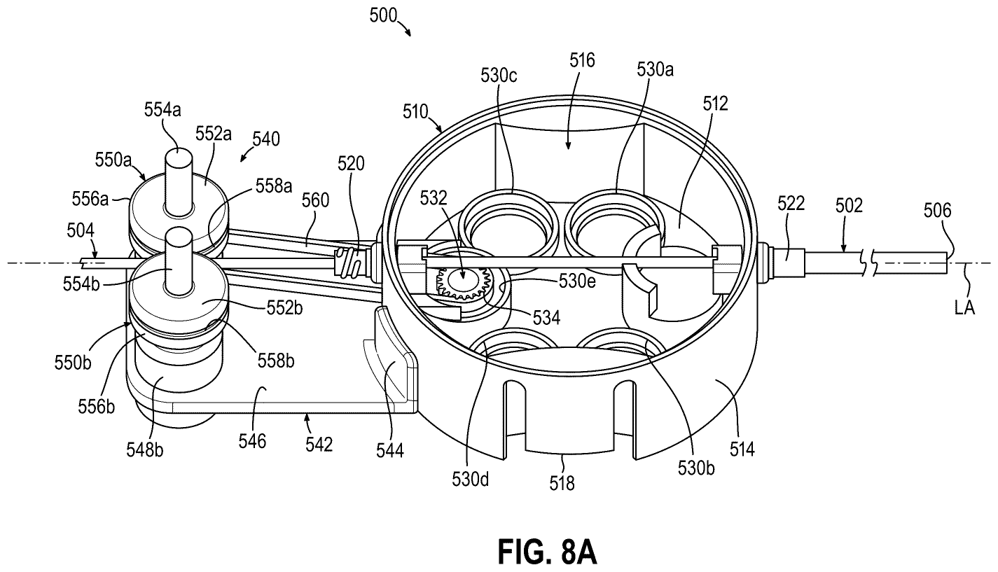

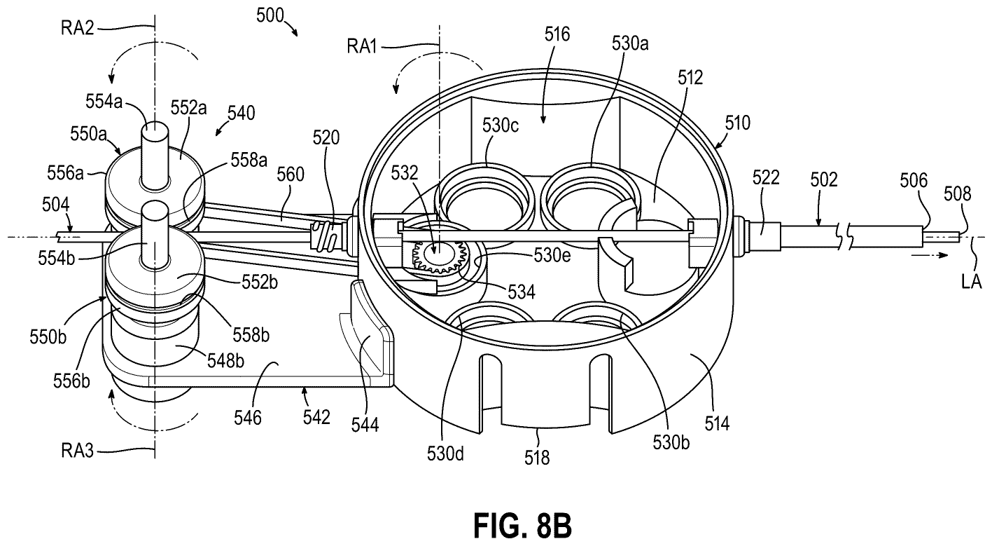

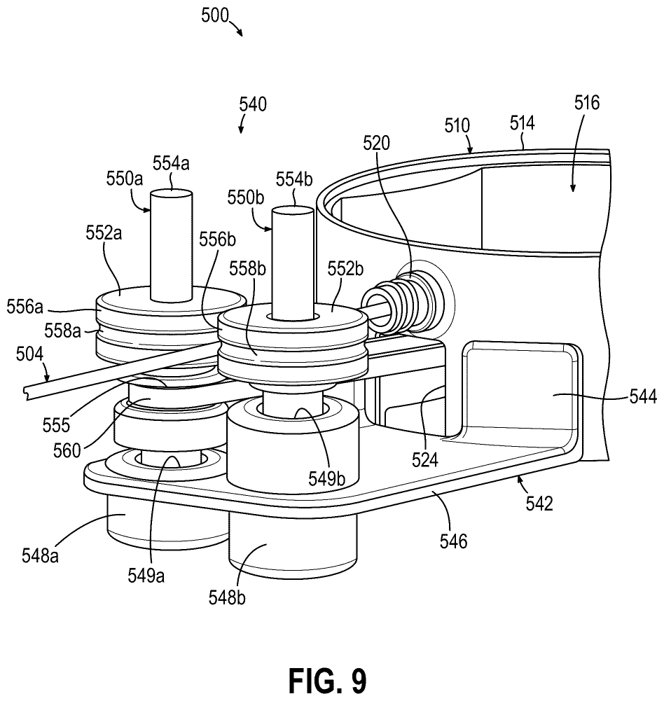

FIGS. 8A-9 show an example of an instrument handle (500) that may be used with robotic surgical system (10) to steer a first elongate member (502) and/or to longitudinally translate a second elongate member (504). By way of example only, first elongate member (502) may represent a variation of scope (40), access sheath (90), or scope (440). Alternatively, first elongate member (502) may take the form of a catheter and/or any other suitable kind of elongate instrument. In the example shown, first elongate member (502) extends distally from instrument handle (500) to a distal end (506). Second elongate member (504) may represent a shaft of a surgical tool, such as a biopsy tool, which may further include an end effector (not shown) at a distal end (508) (FIG. 8B) of second elongate member (504). Alternatively, second elongate member (504) may take the form of a catheter, a scope, a sheath, and/or any other suitable kind of elongate instrument. Instrument handle (500) may be similar to instrument handle (220) described above, except as otherwise described below.

Instrument handle (500) of the present example includes a housing (510) having a baseplate (512) and a substantially cylindrical sidewall (514) extending upwardly from a radially outer periphery of baseplate (512). In some versions, an upper cap (not shown) may be secured to sidewall (514) over baseplate (512), such that a lower surface of the upper cap, an upper surface of baseplate (512), and a radially inner surface of sidewall (514) may collectively define an interior (516) of housing (510). While not shown, a lower surface of baseplate (512) may define an attachment face similar to attachment face (224) and configured to dock to attachment face (204) of IDM (200). Instrument handle (500) also includes a pair of latching mechanisms (518) (one shown) formed on opposing lateral side regions of sidewall (514) and configured to secure instrument handle (500) to IDM (200) when docked.

Housing (510) of the present example also includes a proximal luer (520) extending proximally from a proximal region of sidewall (514), and a distal grip (522) extending distally from a distal region of sidewall (514) such that proximal luer (520) and distal grip (522) are substantially aligned with each other along a central longitudinal axis (LA) (e.g., defined by a proximal portion of first elongate member (502)). In the example shown, distal grip (522) is configured to fixedly secure a proximal end of first elongate member (502) to housing (510), while proximal luer (520) defines a port that is configured to slidably receive second elongate member (504) for permitting passage of second elongate member (504) through sidewall (514) into interior (516) of housing (510) and further into first elongate member (502) from a position that is proximal of housing (510). As shown in FIG. 9, housing (510) also includes a proximal aperture (524) extending through a proximal region of sidewall (514) below proximal luer (520), the purpose of which is described below.

Housing (510) of the present example further includes a plurality of substantially cylindrical bores (530a, 530b, 530c, 530d, 530e) extending through baseplate (512) and configured to rotatably retain respective drive inputs. More particularly, housing (510) includes five bores (530a, 530b, 530c, 530d, 530e): first and second distal bores (530a, 530b) disposed on first and second opposing lateral sides of the longitudinal axis (LA), respectively, and configured to rotatably retain respective distal drive inputs (not shown) similar to drive inputs (228); first and second intermediate bores (530c, 530d) disposed on the first and second opposing lateral sides of the longitudinal axis (LA), respectively, and configured to rotatably retain respective intermediate drive inputs (not shown) similar to drive inputs (228); and a single proximal bore (530e) disposed directly below the longitudinal axis (LA) and configured to rotatably retain a proximal drive input (532).

The drive inputs that are rotatably retained within first and second distal bores (530a, 530b) are configured to engage the corresponding distal drive outputs (208) of IDM (200), while the drive inputs that are rotatably retained within first and second intermediate bores (530c, 530d) are configured to engage the corresponding intermediate drive outputs (208) of IDM (200) in a manner similar to that described above in connection with FIGS. 4-7. In some cases, the drive inputs that are rotatably retained within distal and intermediate bores (530a, 530b, 530c, 530d) may be operatively coupled to distal end (506) of first elongate member (502) via corresponding pull wires (not shown), such that these drive inputs may be configured to selectively deflect distal end (506) of first elongate member (502) laterally and/or vertically away from and/or toward the longitudinal axis (LA) to thereby steer first elongate member (502). For example, the drive inputs that are rotatably retained within distal bores (530a, 530b) may be configured to cooperate with each other to facilitate lateral deflection of distal end (506) while the drive inputs that are rotatably retained within intermediate bores (530c, 530d) may be configured to cooperate with each other to facilitate vertical deflection of distal end (506), or vice versa.

Proximal drive input (532) may be similar to drive inputs (228) described above, except as otherwise described below. In this regard, proximal drive input (532) of the present example is rotatable within proximal bore (530e); and may include a lower receptacle or socket (not shown) that is recessed relative to the attachment face and that is configured to engage the proximal drive output (208) of IDM (200) to facilitate driving of proximal drive input (532). In the example shown, proximal drive input (532) includes an upper gear (534) that is rotatable about a first rotation axis (RA1) that is substantially perpendicular to the longitudinal axis (LA). More particularly, upper gear (534) is configured to rotate about the first rotation axis (RA1) in response to driving of proximal drive input (532) by the proximal drive output (208) of IDM (200).

In this regard, instrument handle (500) of the present example further includes a second elongate member feed assembly (540) positioned proximally relative to housing (510) and configured to convert rotation of upper gear (534) about the first rotation axis (RA1) into translation of second elongate member (504) along the longitudinal axis (LA). In the example shown, second elongate member feed assembly (540) is coupled to housing (510) via a mounting bracket (542). More particularly, mounting bracket (542) includes a pair of flanges (544) fixedly secured to a proximal region of sidewall (514) on opposing lateral sides of proximal aperture (524), and a platform (546) extending proximally from flanges (544) such that platform (546) defines a plane that is substantially parallel to baseplate (512). Mounting bracket (542) of the present example further includes first and second collars (548a, 548b) each extending upwardly and/or downwardly from platform (546), and each defining a respective substantially cylindrical bore (549a, 549b).

In the example shown, second elongate member feed assembly (540) includes first and second roller assemblies (550a, 550b) rotatably supported by first and second collars (548a, 548b), respectively. In this regard, first and second roller assemblies (550a, 550b) of the present example include first and second roller wheels (552a, 552b) fixedly secured to first and second axles (554a, 554b), respectively. As shown, first and second axles (554a, 554b) are rotatably received within first and second bores (549a, 549b), such that first and second axles (554a, 554b) are configured to rotate together with the respective roller wheels (552a, 552b) about second and third rotation axes (RA2, RA3) (e.g., defined by axles (554a, 554b) and/or bores (549a, 549b)), respectively. First roller assembly (550a) of the present example also includes a gear (555) (FIG. 9) fixedly secured to first axle (554a), such that gear (555) is configured to rotate together with first axle (554a) and first roller wheel (552a) about the second rotation axis (RA2).

First and second roller wheels (552a, 552b) of the present example are each substantially disc-shaped, and include respective radially outer surfaces (556a, 556b) and annular grooves (558a, 558b) extending radially inwardly from the respective radially outer surfaces (556a, 556b). Grooves (558a, 558b) are configured to frictionally engage second elongate member (504) to facilitate translation of second elongate member (504) along the longitudinal axis (LA) as described in greater detail below. In some other versions, outer surfaces (556a, 556b) may be configured to frictionally engage second elongate member (504) to facilitate such translation of second elongate member (504), such that grooves (558a, 558b) may be omitted. Outer surfaces (556a, 556b) and/or grooves (558a, 558b) may be textured and/or include any other suitable gripping features to enhance the frictional engagement of each roller wheel (552a, 552b) with second elongate member (504).

While roller assemblies (550a, 550b) of the present example are spaced apart from each other by a fixed distance, roller assemblies (550a, 550b) may alternatively be spaced apart from each other by a variable distance. For example, rather than being rotatably supported on a single mounting bracket (542) that is fixedly secured to housing (510), roller assemblies (550a, 550b) may be rotatably supported on respective mounting brackets, with at least one such mounting bracket being movable relative to housing (510) (and thus relative to the other such mounting bracket). It will be appreciated that spacing roller assemblies (550a, 550b) apart from each other by a variable distance may allow the distance to be adjusted based on an external cross dimension (e.g., diameter) of second elongate member (504) to optimize the frictional engagement of each roller wheel (552a, 552b) with second elongate member (504) for facilitating the translation of second elongate member (504) along the longitudinal axis (LA). In some such cases, roller assemblies (550a, 550b) may be resiliently biased toward each other by one or more springs or other suitable biasing member(s) such that each roller wheel (552a, 552b) may apply a predetermined amount of force to second elongate member (504). In addition, or alternatively, second elongate member feed assembly (540) may include a clutching mechanism or any other suitable mechanism configured to allow the operator (or robotic system (10)) to manually (or automatically) open and close roller wheels (552a, 552b) onto second elongate member (504) and effectively lock roller wheels (552a, 552b) in the closed position (e.g., when roller wheels (552a, 552b) are closed onto second elongate member (504)).

As shown, first roller assembly (550a) is operatively coupled to proximal drive input (532) via a power transmission member in the form of a belt (560) that is configured to drive rotation of first roller assembly (550a) about the second rotation axis (RA2) in response to rotation of proximal drive input (532) about the first rotation axis (RA1). More particularly, belt (560) is looped over upper gear (534) of proximal drive input (532) and gear (555) of first roller assembly (550a), such that belt (560) frictionally engages both gears (534, 555). In this manner, belt (560) may be configured to drive rotation of first roller assembly (550a) about the second rotation axis (RA2) in a direction (e.g., clockwise or counterclockwise) that is the same as that in which proximal drive input (532) is rotating about the first rotation axis (RA1). Thus, first roller wheel (552a) may be referred to as a driven wheel (552a). As shown in FIG. 9, belt (560) extends through proximal aperture (524) of housing (510) in order to loop over both gear (534) disposed within interior (516) of housing (510), and gear (555) disposed outside of housing (510). In some versions, belt (560) may be toothed to enhance the frictional engagement of belt (560) with each gear (534, 555).

While belt (560) is shown in the present example, any other suitable type of transmission member(s) may be used in place of belt (560), such as a chain (e.g., a roller chain) or a gear train, to drive rotation of first roller assembly (550a). Due to the frictional engagement of each roller wheel (552a, 552b) with second elongate member (504), first roller wheel (552a) may be configured to cooperate with second roller wheel (552b) to translate second elongate member (504) along the longitudinal axis (LA) in response to rotation of first roller wheel (552a) as driven by belt (560).

In the example shown, second roller assembly (550b) is not operatively coupled to proximal drive input (532) via its own power transmission member, but rather is configured to rotate about the third rotation axis (RA3) in response to the translation of second elongate member (504) along the longitudinal axis (LA) that is caused by rotation of first roller wheel (552a). For example, second roller assembly (550b) may be configured to rotate about the third rotation axis (RA3) in a direction opposite to that in which first roller assembly (550a) is rotating, in response to such translation of second elongate member (504). Thus, second roller wheel (552a) may be referred to as an idler wheel (552b). In addition, or alternatively, outer surface (556a) of first roller wheel (552a) may frictionally engage outer surface (556b) of second roller wheel (552b) so that second roller assembly (550b) may be configured to rotate about the third rotation axis (RA3) directly in response to rotation of first roller assembly (550a) about the second rotation axis (RA2), without necessarily requiring the presence of second elongate member (504).

In some other versions, second roller assembly (550b) may be operatively coupled to proximal drive input (532) via a suitable power transmission member that is configured to drive rotation of second roller assembly (550b) about the third rotation axis (RA3) in a direction opposite to that in which belt (560) is driving rotation of first roller assembly (550a) about the second rotation axis (RA2), in response to rotation of proximal drive input (532) about the first rotation axis (RA1). For example, second roller assembly (550b) may include a gear (e.g., similar to gear (555)) that is configured to mesh with one or more additional gears (not shown) that are configured to mesh with gear (534) to drive rotation of second roller assembly (550b) about the third rotation axis (RA3) in a direction opposite to that in which proximal drive input (532) is rotating about the first rotation axis (RA1). In such cases, both roller wheels (552a, 552b) may be referred to as driven wheels (552a, 552b).

While axles (554a, 554b) of the present example are configured to rotate together with the respective roller wheels (552a, 552b) about the corresponding rotation axes (RA2, RA3), axles (554a, 554b) may alternatively be fixed against movement relative to mounting bracket (542) and roller wheels (552a, 552b) may be rotatably secured to the respective axles (554a, 554b) such that axles (554a, 554b) may be configured to remain stationary during rotation of the respective roller wheels (552a, 552b) about the corresponding rotation axes (RA2, RA3). In such cases, gear (555) may be fixedly secured to first roller wheel (552a), such that gear (555) may be configured to rotate together with first roller wheel (552a) about the second rotation axis (RA2). For example, gear (555) may be integrally formed together with first roller wheel (552a) as a unitary (e.g., monolithic) piece. Thus, belt (560) may still be configured to drive rotation of first roller wheel (552a) in cases where first axle (554a) is stationary, such that first roller wheel (552a) may still be referred to as a driven wheel (552a).

In use, second elongate member (504) may initially be in a proximally retracted position in which distal end (508) of second elongate member (504) is proximal of distal end (506) of first elongate member (502), as shown in FIG. 8A. For example, distal end (508) of second elongate member (504) may be disposed within a lumen (not shown) of first elongate member (502) when second elongate member (504) is in the proximally retracted position. Proximal drive input (532) including upper gear (534) may then be rotated counterclockwise about the first rotation axis (RA1), such as via the proximal drive output (208) of IDM (200), and belt (560) may respond by driving counterclockwise rotation of first roller wheel (552a) about the second rotation axis (RA2) to thereby translate second elongate member (504) distally along the longitudinal axis (LA) to a distally extended position in which distal end (508) of second elongate member (504) is distal of distal end (506) of first elongate member (502), as shown in FIG. 8B. Such distal translation of second elongate member (504) may cause clockwise rotation of second roller wheel (552b) about the third rotation axis (RA3). In some instances, proximal drive input (532) including upper gear (534) may subsequently be rotated clockwise about the first rotation axis (RA1), such as via the proximal drive output (208) of IDM (200), and belt (560) may respond by driving clockwise rotation of first roller wheel (552a) about the second rotation axis (RA2) to thereby translate second elongate member (504) proximally along the longitudinal axis (LA) to the proximally retracted position. Such proximal translation of second elongate member (504) may cause counterclockwise rotation of second roller wheel (552b) about the third rotation axis (RA3).

While not shown in FIGS. 8A-8B, any one or more of the drive inputs (e.g., like drive inputs (228)) that are rotatably retained within distal and/or intermediate bores (530a, 530b, 530c, 530d) may be rotated about corresponding axes of rotation, such as via the corresponding distal drive outputs (208) of IDM (200), to deflect distal end (506) of first elongate member (502) laterally and/or vertically relative to the longitudinal axis (LA) via the corresponding pull wires to thereby steer first elongate member (502) at any time before, during, or after proximally and/or distally translating second elongate member (504) along the longitudinal axis (LA). Thus, instrument handle (500) may be configured to both longitudinally translate second elongate member (504) through first elongate member (502), and steer first elongate member (502).

### B. Example of Instrument Handle With Internal Elongate Member Feed Assembly

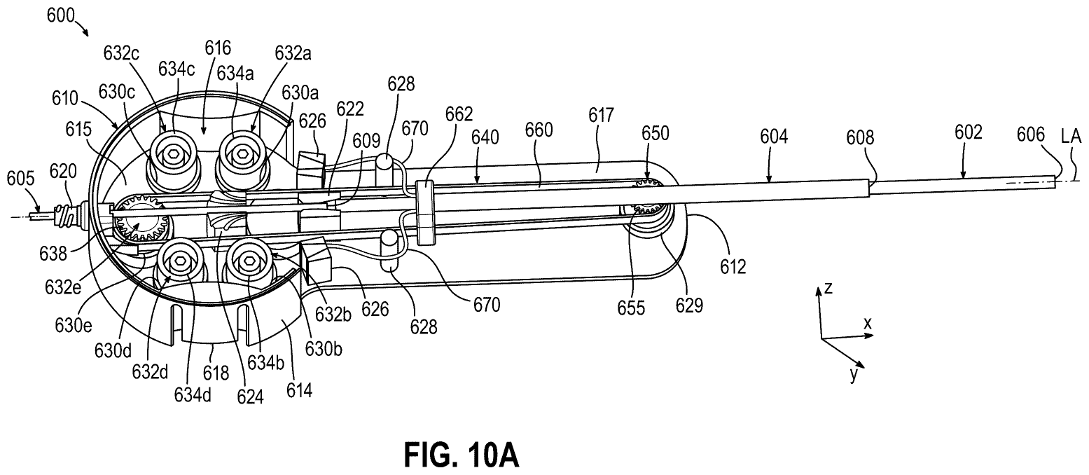

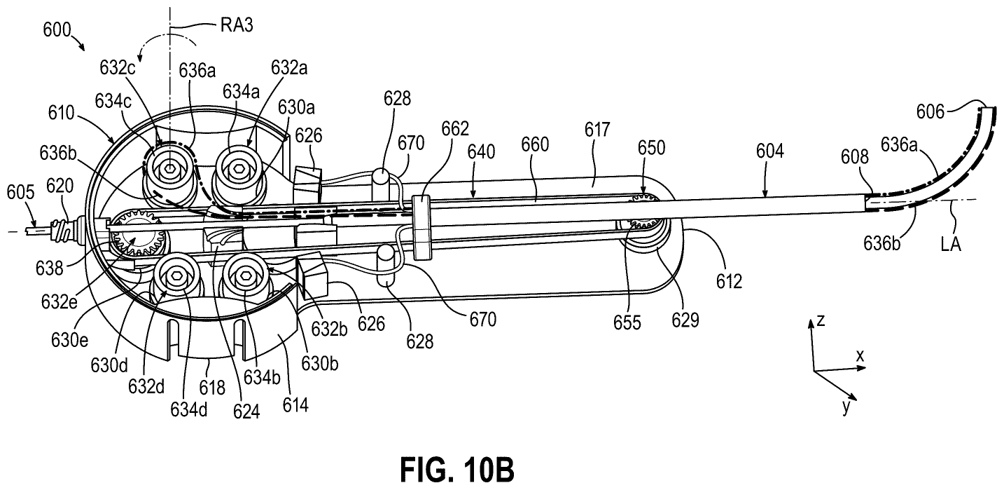

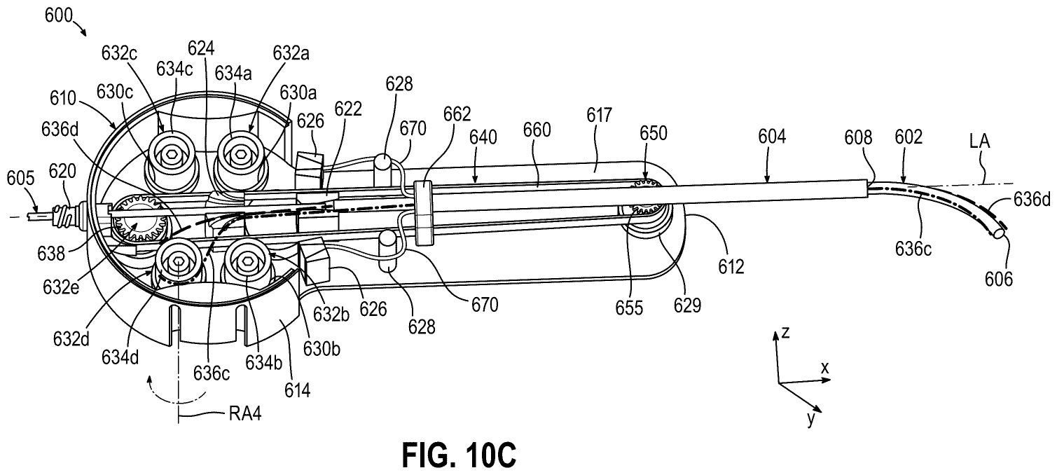

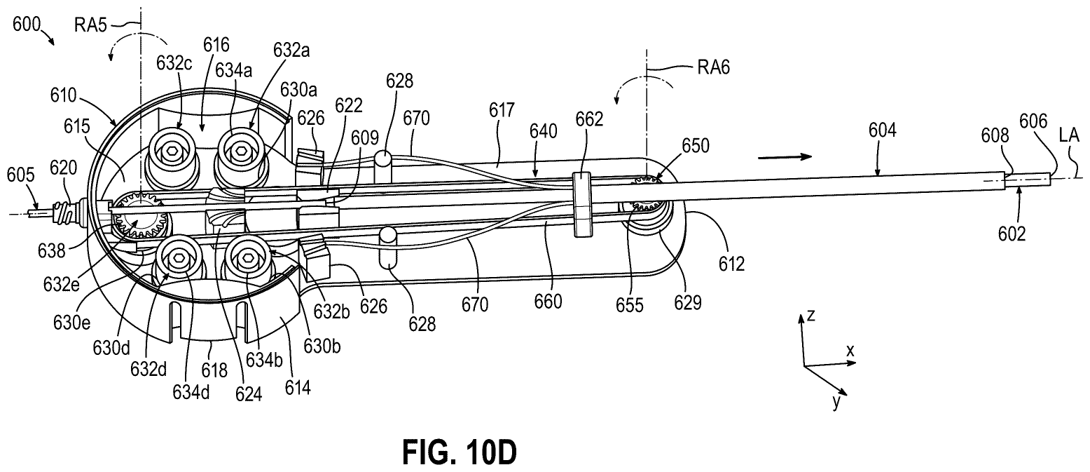

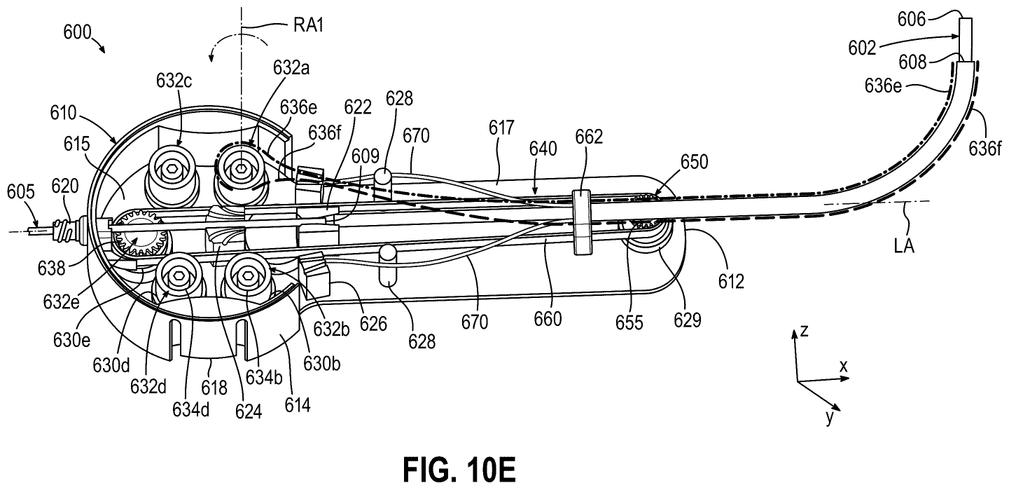

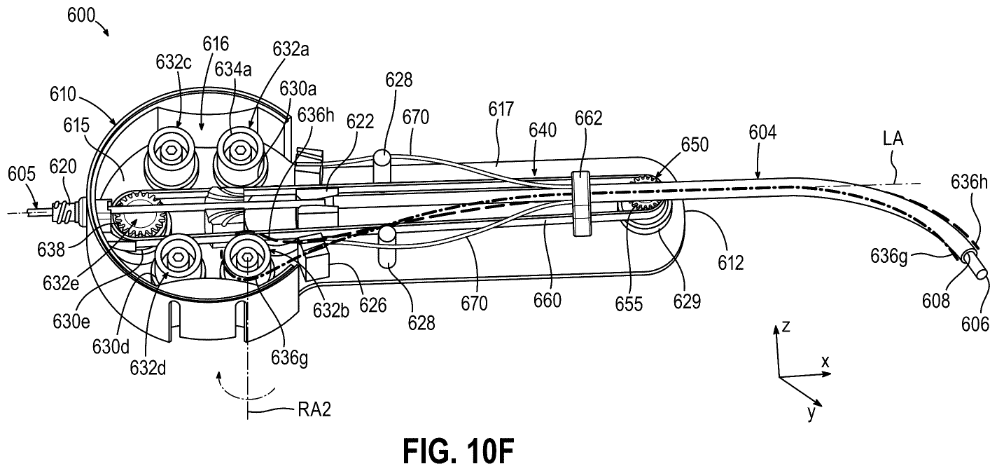

FIGS. 10A-10F show an example of an instrument handle (600) that may be used with robotic surgical system (10) to steer a first elongate member (602), to longitudinally translate and/or steer a second elongate member (604), and/or to facilitate insertion of a third elongate member (605) into first elongate member (602). By way of example only, first elongate member (602) may represent a variation of scope (40) or scope (440). Alternatively, first elongate member (602) may take the form of a catheter, a sheath, and/or any other suitable kind of elongate instrument. In the example shown, first elongate member (602) extends distally from instrument handle (600) to a distal end (606). Second elongate member (604) may represent a variation of access sheath (90). Alternatively, second elongate member (604) may take the form of a catheter, a scope, and/or any other suitable kind of elongate instrument. In the example shown, second elongate member (604) extends distally from instrument handle (600) to a distal end (608). Third elongate member (605) may represent a shaft of a surgical tool, such as a biopsy tool, which may further include an end effector (not shown) at a distal end (609) of third elongate member (605). Alternatively, third elongate member (605) may take the form of a catheter, a scope, a sheath, and/or any other suitable kind of elongate instrument. Instrument handle (600) may be similar to instrument handle (220) described above, except as otherwise described below.

Instrument handle (600) of the present example includes a housing (610) having a baseplate (612) and a substantially C-shaped sidewall (614) extending upwardly from baseplate (612). In the example shown, baseplate (612) includes a substantially circular proximal baseplate portion (615) and a substantially elongate distal baseplate portion (617) extending distally from proximal baseplate portion (615), with sidewall (614) extending upwardly from a radially outer periphery of proximal baseplate portion (615). In some versions, an upper cap (not shown) may be secured to sidewall (614) over baseplate (612), such that a lower surface of the upper cap, an upper surface of baseplate (612), and a radially inner surface of sidewall (614) may collectively define an interior (616) of housing (610). While not shown, a lower surface of baseplate (612) (e.g., a lower surface of proximal baseplate portion (615)) may define an attachment face similar to attachment face (224) and configured to dock to attachment face (204) of IDM (200). Instrument handle (600) also includes a pair of latching mechanisms (618) (one shown) formed on opposing lateral side regions of sidewall (614) and configured to secure instrument handle (600) to IDM (200) when docked.

Housing (610) of the present example also includes a proximal luer (620) extending proximally from a proximal region of sidewall (614), and a distal grip (622) extending upwardly from a distal region of proximal baseplate portion (615) such that proximal luer (620) and distal grip (622) are substantially aligned with each other along a central longitudinal axis (LA) (e.g., defined by a proximal portion of first elongate member (602)). In the example shown, distal grip (622) is configured to fixedly secure a proximal end of first elongate member (602) to housing (610), while proximal luer (620) defines a port that is configured to slidably receive third elongate member (605) for permitting passage of third elongate member (605) through sidewall (614) into interior (616) of housing (610) and further into first elongate member (602) from a position that is proximal of housing (610). As shown, housing (610) also includes a pair of proximal pull wire guides (624) extending upwardly from a substantially middle region of proximal baseplate portion (615) on first and second opposing lateral sides of the longitudinal axis (LA), and a pair of distal pull wire guides (626) extending upwardly from a distal region of proximal portion (615) (and/or from a proximal region of distal baseplate portion (617)) on opposing lateral sides of distal grip (622). Housing (610) also includes a pair of posts (628) extending upwardly from opposing lateral side regions of distal baseplate portion (617), and a collar (629) extending upwardly from a distal region of distal baseplate portion (617) and defining a substantially cylindrical bore (not shown), the purposes of which are described below.

Housing (610) of the present example further includes a plurality of substantially cylindrical bores (630a, 630b, 630c, 630d, 630e) extending through proximal baseplate portion (615) and configured to rotatably retain respective drive inputs (632a, 632b, 632c, 632d, 632e). More particularly, housing (610) includes five bores (630a, 630b, 630c, 630d, 630e): first and second distal bores (630a, 630b) disposed on the first and second opposing lateral sides of the longitudinal axis (LA), respectively, and configured to rotatably retain respective distal drive inputs (632a, 632b); first and second intermediate bores (630c, 630d) disposed on the first and second opposing lateral sides of the longitudinal axis (LA), respectively, and configured to rotatably retain respective intermediate drive inputs (632c, 632d); and a single proximal bore (630e) disposed directly below the longitudinal axis (LA) and configured to rotatably retain a proximal drive input (632e).

Distal and intermediate drive inputs (632a, 632b, 632c, 632d) may be similar to drive inputs (228) described above, except as otherwise described below. In this regard, first and second distal drive input (632a, 632b) of the present example are rotatable within first and second distal bores (630a, 630b), respectively, and may each include a lower receptacle or socket (not shown) that is recessed relative to the attachment face and that is configured to engage the respective distal drive output (208) of IDM (200) to facilitate driving of each distal drive input (632a, 632b). Likewise, first and second intermediate drive input (632c, 632d) of the present example are rotatable within first and second intermediate bores (630c, 630d), respectively, and may each include a lower receptacle or socket (not shown) that is recessed relative to the attachment face and that is configured to engage the respective distal drive output (208) of IDM (200) to facilitate driving of each intermediate drive input (632c, 632d). In the example shown, distal and intermediate drive inputs (632a, 632b, 632c, 632d) include respective upper pulleys (634a, 634b, 634c, 634d) that are rotatable about respective rotation axes (RA1, RA2, RA3, RA4). More particularly, upper pulleys (634a, 634b, 634c, 634d) are configured to rotate about the respective rotation axes (RA1, RA2, RA3, RA4) in response to driving of the respective drive inputs (632a, 632b, 632c, 632d) by the corresponding drive outputs (208) of IDM (200).

Distal and intermediate drive inputs (632a, 632b, 632c, 632d) of the present example are each operatively coupled to a corresponding distal end (606, 608) of first or second elongate member (602, 604) via corresponding pairs of tendons in the form of pull wires (636a, 636b, 636c, 636d, 636e, 636f, 636g 636h) (FIGS. 10B, 10C, 10E, 10F), such that distal and intermediate drive inputs (632a, 632b, 632c, 632d) may be configured to selectively deflect distal ends (606, 608) of elongate members (602, 604) laterally and/or vertically away from and/or toward the longitudinal axis (LA) to thereby steer elongate members (602, 604).

In the arrangement shown, first intermediate drive input (632c) is operatively coupled to upper and lower regions of distal end (606) of first elongate member (602) via first and second pull wires (636a, 636b) (FIG. 10B), respectively, for vertically deflecting distal end (606) along the x-z plane; second intermediate drive input (632d) is operatively coupled to first and second opposing lateral side regions of distal end (606) of first elongate member (602) via third and fourth pull wires (636c, 636d) (FIG. 10C), respectively, for laterally deflecting distal end (606) along the x-y plane; first distal drive input (632a) is operatively coupled to upper and lower regions of distal end (608) of second elongate member (604) via fifth and sixth pull wires (636e, 636f) (FIG. 10E), respectively, for vertically deflecting distal end (608) along the x-z plane; and second distal drive input (632b) is operatively coupled to first and second opposing lateral side regions of distal end (608) of second elongate member (604) via seventh and eighth pull wires (636g, 636h) (FIG. 10F), respectively, for laterally deflecting distal end (608) along the x-y plane.

It will be appreciated that distal and intermediate drive inputs (632a, 632b, 632c, 632d) may be operatively coupled to distal ends (606, 608) in any other suitable arrangement(s) for facilitating lateral and/or vertical deflection of distal ends (606, 608). In the example shown, pull wire guides (624, 626) are configured to slidably receive respective pairs of pull wires (636a, 636b, 636c, 636d, 636e, 636f, 636g 636h) within respective grooves, and to guide the respective pairs of pull wires (636a, 636b, 636c, 636d, 636e, 636f, 636g 636h) out of interior (616) of housing (610) toward the corresponding distal end (606, 608). While pull wires (636a, 636b, 636c, 636d, 636e, 636f, 636g 636h) are shown in the present example, any other suitable type of tendon(s) may be used in place of one or more pull wires (636a, 636b, 636c, 636d, 636e, 636f, 636g 636h), such as a drive band, a single-strand cable, a multi-strand cable, one or more metals, one or more fibers, and/or any other suitable component that is operable to communicate a pulling force along the length of the respective elongate member (602, 604), to thereby provide articulation of the respective elongate member (602, 604), without substantially stretching.

Upper pulleys (634a, 634b, 634c, 634d) may each include a pair of pull wire spool elements (not shown), such as a pair of annular grooves that each extend radially inwardly from a radially outer surface of the respective upper pulley (634a, 634b, 634c, 634d) and that are each configured to receive a respective one of the corresponding pair of pull wires (636a, 636b, 636c, 636d, 636e, 636f, 636g 636h). The pair of pull wire spool elements of each upper pulley (634a, 634b, 634c, 634d) may be configured to wind (e.g., spool or unspool) the corresponding pair of pull wires (636a, 636b, 636c, 636d, 636e, 636f, 636g 636h) in opposite directions about the respective rotation axes (RA1, RA2, RA3, RA4) in response to driving of the respective drive inputs (632a, 632b, 632c, 632d) by the corresponding drive outputs (208) of IDM (200).

For example, a first pull wire spool element of each upper pulley (634a, 634b, 634c, 634d) may be configured to wind (e.g., spool or unspool) the respective one of the corresponding pair of pull wires (636a, 636b, 636c, 636d, 636e, 636f, 636g 636h) about the respective rotation axes (RA1, RA2, RA3, RA4) in a first direction (e.g., clockwise or counterclockwise) that is the same as that in which the respective drive input (632a, 632b, 632c, 632d) is rotating about the respective rotation axis (RA1, RA2, RA3, RA4); while a second pull wire spool element of each upper pulley (634a, 634b, 634c, 634d) may be configured to wind (e.g., unspool or spool) the respective one of the corresponding pair of pull wires (636a, 636b, 636c, 636d, 636e, 636f, 636g 636h) about the respective rotation axes (RA1, RA2, RA3, RA4) in a second direction (e.g., counterclockwise or clockwise) that is opposite the first direction. Thus, each upper pulley (634a, 634b, 634c, 634d) may be configured to spool a first one of the corresponding pair of pull wires (636a, 636b, 636c, 636d, 636e, 636f, 636g 636h) to thereby proximally retract a distal end of the first one of the corresponding pair of pull wires (636a, 636b, 636c, 636d, 636e, 636f, 636g 636h), while simultaneously unspooling a second one of the corresponding pair of pull wires (636a, 636b, 636c, 636d, 636e, 636f, 636g 636h) to thereby distally extend a distal end of the second one of the corresponding pair of pull wires (636a, 636b, 636c, 636d, 636e, 636f, 636g 636h). As described in greater detail below, the simultaneous proximal retraction and distal extension of the distal ends of a pair of pull wires (636a, 636b, 636c, 636d, 636e, 636f, 636g 636h) may cause deflection of the distal end (606, 608) of the corresponding elongate member (602, 604) laterally or vertically away from and/or toward the longitudinal axis (LA), and may thereby facilitate steering of elongate members (602, 604).

Proximal drive input (632e) may be similar to drive inputs (228) described above, except as otherwise described below. In this regard, proximal drive input (632e) of the present example is rotatable within proximal bore (630e); and may include a lower receptacle or socket (not shown) that is recessed relative to the attachment face and that is configured to engage the proximal drive output (208) of IDM (200) to facilitate driving of proximal drive input (632e). In the example shown, proximal drive input (632e) includes an upper gear (638) that is rotatable about a fifth rotation axis (RA5) that is substantially perpendicular to the longitudinal axis (LA). More particularly, upper gear (634) is configured to rotate about the fifth rotation axis (RA5) in response to driving of proximal drive input (632) by the proximal drive output (208) of IDM (200).

In this regard, instrument handle (600) of the present example further includes a second elongate member feed assembly (640) positioned within interior (616) of housing (610) and configured to convert rotation of upper gear (638) about the fifth rotation axis (RA5) into translation of second elongate member (604) along the longitudinal axis (LA).

In the example shown, second elongate member feed assembly (640) includes a distal gear assembly (650) rotatably supported by collar (629). In this regard, distal gear assembly (650) of the present example includes a distal gear (655) fixedly secured to an axle (not shown). The axle may be rotatably received within the bore defined by collar (629), such that the axle may be configured to rotate together with distal gear (655) about a sixth rotation axis (RA6) (e.g., defined by the axle and/or bore).

As shown, distal gear assembly (650) is operatively coupled to proximal drive input (632e) via a power transmission member in the form of a belt (660) that is configured to carry a carriage (662) along the longitudinal axis (LA) in response to rotation of proximal drive input (632e) about the first rotation axis (RA1). More particularly, belt (660) is looped over upper gear (638) of proximal drive input (632e) and gear (655) of distal gear assembly (650), such that belt (660) frictionally engages both gears (638, 655). Carriage (662) is fixedly secured to a proximal end of second elongate member (604) to facilitate translation of second elongate member (604) along the longitudinal axis (LA). Carriage (662) of the present example is also fixedly secured to first and second ends of belt (660) on one lateral side of carriage (662); and is slidable over belt (660) on the other lateral side of carriage (662). For example, one lateral side of carriage (662) may include a pair of bores (not shown) that are configured to fixedly retain the first and send ends of belt (660), while the other lateral side of carriage (662) may include a bore (not shown) that is configured to slidably receive belt (660). In this manner, gear (638) of distal gear assembly (650) may be configured to drive rotation of distal gear assembly (650) about the sixth rotation axis (RA6) in a direction (e.g., clockwise or counterclockwise) that is the same as that in which proximal drive input (632e) is rotating about the fifth rotation axis (RA5), and to further drive rotation of belt (660) in the same direction.

As shown, belt (660) extends between distal grip (622) and each distal pull wire guide (626) in order to loop over both gears (638, 655) disposed within interior (616) of housing (610). In some versions, belt (660) may be toothed to enhance the frictional engagement of belt (660) with each gear (638, 655). While belt (660) is shown in the present example, any other suitable type of transmission member(s) may be used in place of belt (660), such as a chain (e.g., a roller chain), to drive translation of carriage (662)). Due to the fixed securement of carriage (662) with second elongate member (604), carriage (662) may be configured to cooperate with belt (660) to translate second elongate member (604) along the longitudinal axis (LA) in response to rotation of belt (660) as driven by upper gear (638).

While the axle of the present example is configured to rotate together with distal gear (655) about the sixth rotation axis (RA6), the axle may alternatively be fixed against movement relative to distal baseplate portion (617) and distal gear (655) may be rotatably secured to the axle such that the axle may be configured to remain stationary during rotation of distal gear (655) about the sixth rotation axis (RA6).

In the example shown, second elongate member feed assembly (640) further includes a pair of resilient biasing members in the form of coil pipes (670) that are configured to maintain tension in at least some of the pull wires (636a, 636b, 636c, 636d, 636e, 636f, 636g 636h) during translation of second elongate member (604) along the longitudinal axis (LA). For example, coil pipes (670) may be configured to maintain tension in fifth, sixth, seventh, and/or eighth pull wires (636e, 636f, 636g, 636h) that are coupled to distal end (608) of second elongate member (604). In this regard, coil pipes (670) of the present example each extend from a respective one of distal pull wire guides (626) to a respective lateral side of carriage (662). As shown, coil pipes (670) may be disposed laterally outwardly of the respective posts (628) to maintain coil pipes (670) at suitable locations within interior (616) of housing (610), such as to avoid interfering with the routing of pull wires (636a, 636b, 636c, 636d, 636e, 636f, 636g 636h) from the respective upper pulleys (634a, 634b, 634c, 634d) to the respective distal ends (606, 608).

While coil pipes (670) are shown in the present example, any other suitable type of resilient biasing member(s) may be used in place of coil pipes (670) to maintain tension in at least some of the pull wires (636a, 636b, 636c, 636d, 636e, 636f, 636g 636h). For instance, resiliently tensioning idler pulleys may be used to maintain tension in at least some of the pull wires (636a, 636b, 636c, 636d, 636e, 636f, 636g 636h). Such tensioning idler pulleys may be resiliently biased (e.g., via pivot arms, etc.) to bear against pull wires (636a, 636b, 636c, 636d, 636e, 636f, 636g 636h) and thereby maintain tension in pull wires (636a, 636b, 636c, 636d, 636e, 636f, 636g 636h), without impeding translation of pull wires (636a, 636b, 636c, 636d, 636e, 636f, 636g 636h).

In use, second elongate member (604) may initially be in a proximally retracted position in which distal end (608) of second elongate member (604) is proximal of distal end (606) of first elongate member (602), as shown in FIG. 10A. For example, first elongate member (602) may extend distally from a lumen (not shown) of second elongate member (604) when second elongate member (604) is in the proximally retracted position.

As shown in FIG. 10B, first intermediate drive input (632c) including upper pulley (634c) may be rotated counterclockwise about the third rotation axis (RA3), such as via the first intermediate drive output (208) of IDM (200), to spool first pull wire (636a) while unspooling second pull wire (636b), to thereby deflect distal end (606) of first elongate member (602) upwardly (along the x-z plane) relative to the longitudinal axis (LA). While not shown, first intermediate drive input (632c) including upper pulley (634c) may conversely be rotated clockwise about the third rotation axis (RA3), such as via the first intermediate drive output (208) of IDM (200), to unspool first pull wire (636a) while spooling second pull wire (636b), to thereby deflect distal end (606) of first elongate member (602) downwardly (along the x-z plane)relative to the longitudinal axis (LA). [000100] As shown in FIG. 10C, second intermediate drive input (632d) including upper pulley (634d) may be rotated clockwise about the fourth rotation axis (RA4), such as via the second intermediate drive output (208) of IDM (200), to spool third pull wire (636c) while unspooling fourth pull wire (636d) to thereby deflect distal end (606) of first elongate member (602) laterally in a first direction (along the x-y plane) relative to the longitudinal axis (LA). While not shown, second intermediate drive input (632d) including upper pulley (634d) may conversely be rotated counterclockwise about the fourth rotation axis (RA4), such as via the second intermediate drive output (208) of IDM (200), to unspool third pull wire (636c) while spooling fourth pull wire (636d) to thereby deflect distal end (606) of first elongate member (602) laterally in a second direction (along the x-y plane) relative to the longitudinal axis (LA).

As shown in FIG. 10D, proximal drive input (632e) including upper gear (638) may be rotated counterclockwise about the fifth rotation axis (RA5), such as via the proximal drive output (208) of IDM (200), and belt (660) may respond by driving counterclockwise rotation of distal gear (655) about the sixth rotation axis (RA6) and by itself rotating counterclockwise to thereby translate carriage (662) together with second elongate member (604) distally along the longitudinal axis (LA) to a distally extended position in which distal end (608) of second elongate member (604) is distal of distal end (606) of first elongate member (602) and/or closer to distal end (606) than when in the proximally retracted position. In some instances, proximal drive input (632e) including upper gear (638) may subsequently be rotated clockwise about the fifth rotation axis (RA5), such as via the proximal drive output (208) of IDM (200), and belt (660) may respond by driving clockwise rotation of distal gear (655) about the sixth rotation axis (RA6) and by itself rotating counterclockwise to thereby translate carriage (662) together with second elongate member (604) proximally along the longitudinal axis (LA) to the proximally retracted position.

As shown in FIG. 10E, first distal drive input (632a) including upper pulley (634a) may be rotated counterclockwise about the first rotation axis (RA1), such as via the first distal drive output (208) of IDM (200), to spool fifth pull wire (636e) while unspooling sixth pull wire (636f), to thereby deflect distal end (608) of second elongate member (604) upwardly (along the x-z plane) relative to the longitudinal axis (LA). While not shown, first distal drive input (632a) including upper pulley (634a) may conversely be rotated clockwise about the first rotation axis (RA1), such as via the first distal drive output (208) of IDM (200), to unspool fifth pull wire (636e) while spooling sixth pull wire (636f), to thereby deflect distal end (608) of second elongate member (604) downwardly (along the x-z plane) relative to the longitudinal axis (LA).

In the example shown, distal end (606) of first elongate member (602) is vertically deflected together with distal end (608) of second elongate member (604). In this regard, rotation of first distal drive input (632a) about the first rotation axis (RA1) may be synchronized with an appropriate corresponding rotation of first intermediate drive input (632c) about the third rotation axis (RA3) to achieve simultaneous vertical deflection of the distal ends (606, 608) of both elongate members (602, 604). In addition, or alternatively, one of first or second elongate members (602, 604) (e.g., second elongate member (604)) may have a stiffness that is sufficiently greater than that of the other of first or second elongate members (602, 604) (e.g., first elongate member (602)) to impart a curvature to the other of first or second elongate members (602, 604), such that the vertical deflection of the distal end (606, 608) of the stiffer elongate member (602, 604) may cause a corresponding vertical deflection of the distal end (606, 608) of the less stiff elongate member (602, 604). In some such cases, rotation of the drive input (632a, 632c) of the stiffer elongate member (602, 604) about the respective rotation axis (RA1, RA3) may be actively driven by the corresponding drive output (208), while the drive input (632a, 632c) of the less stiff elongate member (602, 604) may be permitted to freely rotate about the respective rotation axis (RA1, RA3) by the corresponding drive output (208) to accommodate the vertical deflection of the distal end (606, 608) of the less stiff elongate member (602, 604). In this manner, the drive input (632a, 632c) of the stiffer elongate member (602, 604) may effectively control the vertical deflection of the distal ends (606, 608) of both elongate members (602, 604).

As shown in FIG. 10F, second distal drive input (632b) including upper pulley (634b) may be rotated clockwise about the second rotation axis (RA2), such as via the second distal drive output (208) of IDM (200), to spool seventh pull wire (636g) while unspooling eighth pull wire (636h), to thereby deflect distal end (608) of second elongate member (604) laterally in the first direction (along the x-y plane) relative to the longitudinal axis (LA). While not shown, second distal drive input (632b) including upper pulley (634b) may conversely be rotated counterclockwise about the second rotation axis (RA2), such as via the second distal drive output (208) of IDM (200), to unspool seventh pull wire (636g) while spooling eighth pull wire (636h), to thereby deflect distal end (608) of second elongate member (604) laterally in the second direction (along the x-y plane) relative to the longitudinal axis (LA).

In the example shown, distal end (606) of first elongate member (602) is laterally deflected together with distal end (608) of second elongate member (604). In this regard, rotation of second distal drive input (632b) about the second rotation axis (RA2) may be synchronized with an appropriate corresponding rotation of second intermediate drive input (632d) about the fourth rotation axis (RA4) to achieve simultaneous lateral deflection of the distal ends (606, 608) of both elongate members (602, 604). In addition, or alternatively, one of first or second elongate members (602, 604) (e.g., second elongate member (604)) may have a stiffness that is sufficiently greater than that of the other of first or second elongate members (602, 604) (e.g., first elongate member (602)) to impart a curvature to the other of first or second elongate members (602, 604), such that the lateral deflection of the distal end (606, 608) of the stiffer elongate member (602, 604) may cause a corresponding lateral deflection of the distal end (606, 608) of the less stiff elongate member (602, 604). In some such cases, rotation of the drive input (632b, 632d) of the stiffer elongate member (602, 604) about the respective rotation axis (RA2, RA4) may be actively driven by the corresponding drive output (208), while the drive input (632b, 632d) of the less stiff elongate member (602, 604) may be permitted to freely rotate about the respective rotation axis (RA2, RA4) by the corresponding drive output (208) to accommodate the lateral deflection of the distal end (606, 608) of the less stiff elongate member (602, 604). In this manner, the drive input (632b, 632d) of the stiffer elongate member (602, 604) may effectively control the lateral deflection of the distal ends (606, 608) of both elongate members (602, 604).

It will be appreciated that any one or more of drive inputs (632a, 632b, 632c, 632d) may be rotated about corresponding axes of rotation (RA1, RA2, RA3, RA4), such as via the corresponding drive outputs (208) of IDM (200), to deflect distal end (606, 608) of one or both elongate members (602, 604) laterally and/or vertically relative to the longitudinal axis (LA) via the corresponding pull wires (636a, 636b, 636c, 636d, 636e, 636f, 636g, 636h) to thereby steer one or both elongate members (602, 604) at any time before, during, or after proximally and/or distally translating second elongate member (604) along the longitudinal axis (LA). Thus, instrument handle (600) may be configured to longitudinally translate second elongate member (604) over first elongate member (602), and steer both first and second elongate members (602, 604).

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: a housing; a first elongate member extending distally from the housing to a first distal end, a proximal portion of the first elongate member defining a longitudinal axis; at least one first drive input rotatably retained by the housing, the at least one first drive input being configured to deflect the first distal end relative to the longitudinal axis; a second elongate member; a second drive input rotatably retained by the housing; and a feed assembly configured to translate the second elongate member along the longitudinal axis in response to rotation of the second drive input.

### Example 2

The apparatus of Example 1, the second drive input being proximal of the at least one first drive input.

### Example 3

The apparatus of any of Examples 1 through 2, the second drive input being configured to rotate about a rotation axis that is substantially perpendicular to the longitudinal axis.

### Example 4

The apparatus of any of Examples 1 through 3, the second drive input including a gear.

### Example 5

The apparatus of any of Examples 1 through 4, the feed assembly including a gear configured to rotate in response to rotation of the second drive input.

### Example 6

The apparatus of Example 5, the feed assembly including a transmission member configured to drive rotation of the gear in response to rotation of the second drive input.

### Example 7

The apparatus of Example 6, the transmission member including a belt.

### Example 8

The apparatus of any of Examples 1 through 7, the feed assembly including a pair of roller wheels configured to frictionally engage the second elongate member, at least one roller wheel of the pair of roller wheels being configured to rotate in response to rotation of the second drive input to thereby translate the second elongate member along the longitudinal axis.

### Example 9

The apparatus of any of Examples 1 through 7, the feed assembly including a carriage fixedly secured to the second elongate member, the carriage being configured to translate in response to rotation of the second drive input to thereby translate the second elongate member along the longitudinal axis.

### Example 10

The apparatus of any of Examples 1 through 9, the second elongate member having a second distal end, the apparatus further comprising at least one third drive input rotatably retained by the housing, the at least one third drive input being configured to deflect the second distal end for deflecting the second distal end relative to the longitudinal axis.

### Example 11

The apparatus of any of Examples 1 through 10, one of the first or second elongate members being disposed within the other of the first or second elongate members.

### Example 12

The apparatus of Example 11, the first elongate member being disposed within the second elongate member.

### Example 13

The apparatus of Example 11, the second elongate member being disposed within the first elongate member.

### Example 14

The apparatus of any of Examples 1 through 13, the feed assembly being proximal of the housing.

### Example 15

The apparatus of any of Examples 1 through 13, the feed assembly being disposed within an interior of the housing.

### Example 16

The apparatus of any of Examples 1 through 15, the at least one first drive input including at least one pulley.

### Example 17

The apparatus of any of Examples 1 through 16, the at least one first drive input being operatively coupled to the first distal end via at least one tendon.

### Example 18

The apparatus of any of Examples 1 through 17, the first elongate member including a scope.

### Example 19

The apparatus of any of Examples 1 through 18, the second elongate member including at least one of a sheath or a tool shaft.

### Example 20

The apparatus of Example 19, the second elongate member including a biopsy tool shaft.

### Example 21

The apparatus of any of Examples 1 through 20, the housing being configured to releasably dock to an instrument device manipulator, the at least one first drive input being configured to rotate about at least one first rotation axis in response to rotation of at least one first drive output of the instrument device manipulator about the at least one first rotation axis, the second drive input being configured to rotate about a second rotation axis in response to rotation of a second drive output of the instrument device manipulator about the second rotation axis.

### Example 22

The apparatus of Example 21, the second rotation axis intersecting the longitudinal axis and/or being non-parallel relative to the longitudinal axis.

### Example 23

The apparatus of any of Examples 21 through 22, the second rotation axis being substantially parallel to the first rotation axis.

### Example 24

The apparatus of any of Examples 21 through 23, the second rotation axis being spaced apart from the first rotation axis by a fixed distance.

### Example 25

The apparatus of any of Examples 1 through 24, the housing including a baseplate and a plurality of bores extending through the baseplate, the at least one first drive input and the second drive input being rotatably retained within respective bores of the plurality of bores.

### Example 26

The apparatus of any of Examples 1 through 25, the at least one first drive input and the second drive input being at least partially disposed within an interior of the housing.

### Example 27

The apparatus of any of Examples 1 through 26, the housing including a sidewall that is at least one of substantially cylindrical or substantially C-shaped, the at least one first drive input and the second drive input being disposed radially inwardly of the sidewall.

### Example 28

The apparatus of any of Examples 1 through 27, the feed assembly being mounted to the housing.

### Example 29

The apparatus of any of Examples 21 through 28, further comprising the instrument device manipulator, the instrument device manipulator including the at least one first drive output and the second drive output.

### Example 30

The apparatus of Example 29, the housing being releasably docked to the instrument device manipulator.

### Example 31

An apparatus comprising: a housing; a first elongate member extending distally from the housing to a distal end, a proximal portion of the first elongate member defining a longitudinal axis; at least one first drive input rotatably retained by the housing, the at least one first drive input being configured to deflect the distal end relative to the longitudinal axis; a second drive input rotatably retained by the housing; and a pair of roller wheels coupled to the housing, each roller wheel of the pair of roller wheels being configured to frictionally engage a second elongate member, at least one roller wheel of the pair of roller wheels being configured to rotate in response to rotation of the second drive input to thereby translate the second elongate member along the longitudinal axis.

### Example 32

The apparatus of Example 31, the at least one roller wheel of the pair of roller wheels being operatively coupled to the second drive input via a transmission member, the transmission member extending through an aperture in the housing.

### Example 33

The apparatus of Example 32, the transmission member including a belt.

### Example 34

The apparatus of any of Examples 32 through 33, further comprising a gear fixedly secured to the at least one roller wheel of the pair of roller wheels, the transmission member being configured to frictionally engage the gear to thereby rotate the at least one roller wheel of the pair of roller wheels in response to rotation of the second drive input.

### Example 35

The apparatus of any of Examples 32 through 34, the second drive input including a gear configured to frictionally engage the transmission member to thereby drive rotation of the transmission member in response to rotation of the second drive input.

### Example 36

The apparatus of any of Examples 31 through 35, further comprising the second elongate member, the second elongate member being slidably disposed within the first elongate member.

### Example 37

The apparatus of Example 36, the second elongate member including a tool shaft.

### Example 38

The apparatus of Example 37, the tool shaft including a biopsy tool shaft.

### Example 39

The apparatus of any of Examples 31 through 38, the at least one first drive input including a plurality of first drive inputs.

### Example 40

The apparatus of Example 39, the plurality of first drive inputs including four drive inputs.

### Example 41

An apparatus comprising: a housing; a first elongate member extending distally from the housing to a first distal end, a proximal portion of the first elongate member defining a longitudinal axis; at least one first drive input rotatably retained by the housing, the at least one first drive input being configured to deflect the first distal end relative to the longitudinal axis; a second elongate member slidably disposed over the first elongate member; a second drive input rotatably retained by the housing; and a carriage fixedly secured to the second elongate member, the carriage being configured to translate in response to rotation of the second drive input to thereby translate the second elongate member along the longitudinal axis.

### Example 42

The apparatus of Example 41, the second elongate member having a second distal end, the apparatus further comprising at least one third drive input rotatably retained by the housing, the at least one third drive input being configured to deflect the second distal end relative to the longitudinal axis.

### Example 43

The apparatus of any of Examples 41 through 42, the carriage being operatively coupled to the second drive input via a transmission member.

### Example 44

The apparatus of Example 43, the transmission member including a belt.

### Example 45

The apparatus of any of Examples 43 through 44, the carriage being fixedly secured to first and second ends of the transmission member.

### Example 46

The apparatus of Example 45, the carriage being slidable over a portion of the transmission member.

### Example 47

The apparatus of any of Examples 44 through 46, the second drive input including a gear configured to frictionally engage the transmission member to thereby drive rotation of the transmission member in response to rotation of the second drive input.

### Example 48

The apparatus of any of Examples 41 through 47, further comprising a third elongate member slidably disposed within the first elongate member.

### Example 49

The apparatus of Example 48, the third elongate member including a tool shaft.

### Example 50

The apparatus of Example 49, the tool shaft including a biopsy tool shaft.

### Example 51

An apparatus comprising: a housing configured to releasably dock to an instrument device manipulator; a first elongate member extending distally from the housing to a first distal end, a proximal portion of the first elongate member defining a longitudinal axis; at least one first drive input rotatably retained by the housing such that the at least one first drive input is configured to rotate about at least one first rotation axis in response to rotation of at least one first drive output of the instrument device manipulator about the at least one first rotation axis, the at least one first drive input being operatively coupled to the first distal end for deflecting the first distal end relative to the longitudinal axis; a second elongate member having a second distal end, the second elongate member being configured to translate along the longitudinal axis; a second drive input rotatably retained by the housing such that the second drive input is configured to rotate about a second rotation axis in response to rotation of a second drive output of the instrument device manipulator about the second rotation axis; and a feed assembly configured to translate the second elongate member along the longitudinal axis in response to rotation of the second drive input about the second rotation axis.

### Example 52

The apparatus of Example 51, the second drive input being proximal of the at least one first drive input.

### Example 53

The apparatus of any of Examples 51 through 52, the second rotation axis being substantially perpendicular to the longitudinal axis.

### Example 54

The apparatus of any of Examples 51 through 53, the second drive input including a gear.

### Example 55

The apparatus of any of Examples 51 through 54, the feed assembly including a gear configured to rotate in response to rotation of the second drive input.

### Example 56

The apparatus of Example 55, the feed assembly including a transmission member configured to drive rotation of the gear in response to rotation of the second drive input.

### Example 57

The apparatus of Example 56, the transmission member including a belt.

### Example 58

The apparatus of any of Examples 51 through 57, the feed assembly including a pair of roller wheels configured to frictionally engage the second elongate member, at least one roller wheel of the pair of roller wheels being configured to rotate in response to rotation of the second drive input to thereby translate the second elongate member along the longitudinal axis.

### Example 59

The apparatus of any of Examples 51 through 57, the feed assembly including a carriage fixedly secured to the second elongate member, the carriage being configured to translate in response to rotation of the second drive input to thereby translate the second elongate member along the longitudinal axis.

### Example 60

The apparatus of any of Examples 51 through 59, further comprising at least one third drive input rotatably retained by the housing, the at least one third drive input being operatively coupled to the second distal end for deflecting the second distal end relative to the longitudinal axis.

### Example 61

The apparatus of any of Examples 51 through 60, one of the first or second elongate members being disposed within the other of the first or second elongate members.

### Example 62

The apparatus of Example 61, the first elongate member being disposed within the second elongate member.

### Example 63

The apparatus of Example 61, the second elongate member being disposed within the first elongate member.

### Example 64

The apparatus of any of Examples 51 through 63, the feed assembly being proximal of the housing.

### Example 65

The apparatus of any of Examples 51 through 63, the feed assembly being disposed within an interior of the housing.

### Example 66

An apparatus comprising: a housing configured to releasably dock to an instrument device manipulator; a first elongate member extending distally from the housing to a distal end, a proximal portion of the first elongate member defining a longitudinal axis; at least one first drive input rotatably retained by the housing such that the at least one first drive input is configured to rotate about at least one first rotation axis in response to rotation of at least one first drive output of the instrument device manipulator about the at least one first rotation axis, the at least one first drive input being operatively coupled to the distal end for deflecting the distal end relative to the longitudinal axis; a second drive input rotatably retained by the housing such that the second drive input is configured to rotate about a second rotation axis in response to rotation of a second drive output of the instrument device manipulator about the second rotation axis; and a pair of roller wheels coupled to the housing, each roller wheel of the pair of roller wheels being configured to frictionally engage a second elongate member, at least one roller wheel of the pair of roller wheels being configured to rotate in response to rotation of the second drive input about the second rotation axis to thereby translate the second elongate member along the longitudinal axis.

### Example 67

The apparatus of Example 66, the at least one roller wheel of the pair of roller wheels being operatively coupled to the second drive input via a transmission member, the transmission member extending through an aperture in the housing.

### Example 68

The apparatus of any of Examples 66 through 67, further comprising the second elongate member, the second elongate member being slidably disposed within the first elongate member.

### Example 69

An apparatus comprising: a housing configured to releasably dock to an instrument device manipulator; a first elongate member extending distally from the housing to a first distal end, a proximal portion of the first elongate member defining a longitudinal axis; at least one first drive input rotatably retained by the housing such that the at least one first drive input is configured to rotate about at least one first rotation axis in response to rotation of at least one first drive output of the instrument device manipulator about the at least one first rotation axis, the at least one first drive input being operatively coupled to the first distal end for deflecting the first distal end relative to the longitudinal axis; a second elongate member slidably disposed over the first elongate member; a second drive input rotatably retained by the housing such that the second drive input is configured to rotate about a second rotation axis in response to rotation of a second drive output of the instrument device manipulator about the second rotation axis; and a carriage fixedly secured to the second elongate member, the carriage being configured to translate in response to rotation of the second drive input to thereby translate the second elongate member along the longitudinal axis.

### Example 70

The apparatus of Example 69, further comprising at least one third drive input rotatably retained by the housing, the at least one third drive input being operatively coupled to a second distal end of the second elongate member for deflecting the second distal end relative to the longitudinal axis.

### Example 71

A method comprising: releasably docking a housing of an apparatus to an instrument device manipulator; deflecting a first distal end of a first elongate member of the apparatus relative to a longitudinal axis defined by a proximal portion of the first elongate member, the first elongate member extending distally from the housing to the first distal end, the act of deflecting including rotating at least one first drive output of the instrument device manipulator about at least one first rotation axis to thereby rotate at least one first drive input rotatably retained by the housing about the at least one first rotation axis, the at least one first drive input being operatively coupled to the first distal end; and translating a second elongate member of the apparatus along the longitudinal axis, the act of translating including rotating a second drive output of the instrument device manipulator about a second rotation axis to thereby rotate a second drive input rotatably retained by the housing about the second rotation axis such that a feed assembly translates the second elongate member along the longitudinal axis in response to rotation of the second drive input about the second rotation axis.

### Example 72

A method comprising: releasably docking a housing of an apparatus to an instrument device manipulator; deflecting a first distal end of a first elongate member of the apparatus relative to a longitudinal axis defined by a proximal portion of the first elongate member, the first elongate member extending distally from the housing to the first distal end, the act of deflecting including rotating at least one first drive output of the instrument device manipulator about at least one first rotation axis to thereby rotate at least one first drive input rotatably retained by the housing about the at least one first rotation axis, the at least one first drive input being operatively coupled to the first distal end; and translating a second elongate member of the apparatus along the longitudinal axis, the act of translating including rotating a second drive output of the instrument device manipulator about a second rotation axis to thereby rotate a second drive input rotatably retained by the housing about the second rotation axis such that a pair of roller wheels coupled to the housing translates the second elongate member along the longitudinal axis in response to rotation of the second drive input about the second rotation axis.

### Example 73

A method comprising: releasably docking a housing of an apparatus to an instrument device manipulator; deflecting a first distal end of a first elongate member of the apparatus relative to a longitudinal axis defined by a proximal portion of the first elongate member, the first elongate member extending distally from the housing to the first distal end, the act of deflecting including rotating at least one first drive output of the instrument device manipulator about at least one first rotation axis to thereby rotate at least one first drive input rotatably retained by the housing about the at least one first rotation axis, the at least one first drive input being operatively coupled to the first distal end; and translating a second elongate member of the apparatus along the longitudinal axis, the act of translating including rotating a second drive output of the instrument device manipulator about a second rotation axis to thereby rotate a second drive input rotatably retained by the housing about the second rotation axis such that a carriage fixedly secured to the second elongate member translates the second elongate member along the longitudinal axis in response to rotation of the second drive input about the second rotation axis.

### V. Miscellaneous

Versions described above may be designed to be disposed of after a single use, or they may be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that may penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
a housing;
a first elongate member extending distally from the housing to a first distal end, a proximal portion of the first elongate member defining a longitudinal axis;
at least one first drive input rotatably retained by the housing, the at least one first drive input being configured to deflect the first distal end relative to the longitudinal axis;
a second elongate member;
a second drive input rotatably retained by the housing ; and
a feed assembly configured to translate the second elongate member along the longitudinal axis in response to rotation of the second drive input.

2. The apparatus of claim 1, the second drive input being proximal of the at least one first drive input.

3. The apparatus of claim 1, the second drive input being configured to rotate about a rotation axis that is substantially perpendicular to the longitudinal axis.

4. The apparatus of claim 1, the second drive input including a gear.

5. The apparatus of claim 1, the feed assembly including a gear configured to rotate in response to rotation of the second drive input.

6. The apparatus of claim 5, the feed assembly including a transmission member configured to drive rotation of the gear in response to rotation of the second drive input.

7. The apparatus of claim 6, the transmission member including a belt.

8. The apparatus of claim 1, the feed assembly including a pair of roller wheels configured to frictionally engage the second elongate member, at least one roller wheel of the pair of roller wheels being configured to rotate in response to rotation of the second drive input to thereby translate the second elongate member along the longitudinal axis.

9. The apparatus of claim 1, the feed assembly including a carriage fixedly secured to the second elongate member, the carriage being configured to translate in response to rotation of the second drive input to thereby translate the second elongate member along the longitudinal axis.

10. The apparatus of claim 1, the second elongate member having a second distal end, the apparatus further comprising at least one third drive input rotatably retained by the housing, the at least one third drive input being configured to deflect the second distal end relative to the longitudinal axis.

11. The apparatus of claim 1, one of the first or second elongate members being disposed within the other of the first or second elongate members.

12. The apparatus of claim 11, the first elongate member being disposed within the second elongate member.

13. The apparatus of claim 11, the second elongate member being disposed within the first elongate member.

14. The apparatus of claim 1, the feed assembly being proximal of the housing.

15. The apparatus of claim 1, the feed assembly being disposed within an interior of the housing.

16. An apparatus comprising:
a housing;
a first elongate member extending distally from the housing to a distal end, a proximal portion of the first elongate member defining a longitudinal axis;
at least one first drive input rotatably retained by the housing, the at least one first drive input being configured to deflect the distal end relative to the longitudinal axis;
a second drive input rotatably retained by the housing; and
a pair of roller wheels coupled to the housing, each roller wheel of the pair of roller wheels being configured to frictionally engage a second elongate member, at least one roller wheel of the pair of roller wheels being configured to rotate in response to rotation of the second drive input to thereby translate the second elongate member along the longitudinal axis.

17. The apparatus of claim 16, the at least one roller wheel of the pair of roller wheels being operatively coupled to the second drive input via a transmission member, the transmission member extending through an aperture in the housing.

18. The apparatus of claim 16, further comprising the second elongate member, the second elongate member being slidably disposed within the first elongate member.

19. An apparatus comprising:
a housing;
a first elongate member extending distally from the housing to a first distal end, a proximal portion of the first elongate member defining a longitudinal axis;
at least one first drive input rotatably retained by the housing, the at least one first drive input being configured to deflect the first distal end relative to the longitudinal axis;
a second elongate member slidably disposed over the first elongate member;
a second drive input rotatably retained by the housing; and
a carriage fixedly secured to the second elongate member, the carriage being configured to translate in response to rotation of the second drive input to thereby translate the second elongate member along the longitudinal axis.

20. The apparatus of claim 19, the second elongate member having a second distal end, the apparatus further comprising at least one third drive input rotatably retained by the housing, the at least one third drive input being configured to deflect the second distal end relative to the longitudinal axis.
